SEQUENCE LISTING (30 sequences)

SEQ ID NO: 1 - DNA, 1503 bp, Bombyx mori
atggacgcgaaccagtttcgggaattcggcagagcagtcatcgatatgttggcgagctacgctgaaaacataagggattatgatgttctgccgtctgttgaacctggttacttgttaagagctctgcctgaaagtgcacccgaacaaccggaggattggaaagacataatgaaagatttcaatcaatcaataatgccgggtgtaacacactggcaatctccgcagttccatgcattctatccttctggctcctcattcgcgagtattataggaaatatgcttagtgacggcttggcggttgtgggatttagttggatggctagccctgcgtgtacagaactggaggtggtcacgatgaattggcttggtaagctattagatttacctgaagaatttctaaattgctcttcgggtcccggaggcggagttattcaaggatcagcgagcgaagccaccttagttggattgcttgtagccaaagataaaactgttcgccgtttcatgaataataatccagatctcgatgaaaacgagataaaagcaaaacttgtagcttatacatccgaccaatgtaactcgtcagtagaaaaagccggcttacttggttctatgaaaatgaaactgttgaaagcggatgctgatggatgcctacgcggagaaacattgaaaagggcaatcgaagaagataagtcgcaaggacttataccctgctatgtcgtcgctaatctgggaacaactggaacttgtgcattcgatcctttacatgaattagggccaatatgtagtgaggaagatatatggcttcacgtagatgcagcctatgcgggagcagcatttttgtgtcctgaatacagacacctaatgaaaggtattgaacattctcaatcgttcgtaacgaatgcacataagtggctaccggttaatttcgattgctctgctatgtgggttaaaaatggttatgatataacgagagcattcgatgtacaaagaatttatttggatgatgtaaaaacgacaatcaagatcccagattacagacactggcaaatgccactaggccgtcgctttagagccttaaaattgtggacagttatgagaatttatggtgctgaaggtttgaaaacgcatatcagacagcaaatagaattagcacagtattttgcaaaactggtacgtgcggacgaacgttttgtgattgggcccgaaccgactatggcattggtctgttttagactgaaagacggtgacacaattacgcgacaattgttggaaaatataacgcaaaaaaagaaagtgtttatggtggccggaacgcacagggatagatacgtcattagattcgtgatctgctcccgattgactaagaaggaagacgtcgattacagctggagccaaataaagaaagaaaccgacctcatctattcagataaaatacacaataaagcacaaataccagctcttgaacaattcacttcaagagaactatgcgaaaaatctaagtaa

SEQ ID NO: 2 - DNA, 1506 bp, Bombyx mori
atggacgcgaaccagtttcgggaattcggcagagcagtcatcgatatgttggcgagctacgctgaaaacataagggattatgatgttctgccgtctgttgaacctggttacttgttaagagctctgcctgaaagtgcacccgaacaaccggaggattggaaagacataatgaaagatttcaatcaatcaataatgccgggtgtaacacactggcaatctccgcagttccatgcattctatccttctggctcctcattcgcgagtattataggaaatatgcttagtgacggcttggcggttgtgggatttagttggatggctagccctgcgtgtacagaactggaggtggtcacgatgaattggcttggtaagctattagatttacctgaagaatttctaaattgctcttcgggtcccggaggcggagttattcaaggatcagcgagcgaagccaccttagttggattgcttgtagccaaagataaaactgttcgccgtttcatgaataataatccagatctcgatgaaaacgagataaaagcaaaacttgtagcttatacatccgaccaatgtcactcgtcagtagaaaaagccggcttacttggttctatgaaaatgaaactgttgaaagcggatgctgatggatgcctacgcggagaaacattgaaaagggcaatcgaagaagataagtcgcaaggacttataccctgctatgtcgtcgctaatctgggaacaactggaacttgtgcattcgatcctttacatgaattagggccaatatgtagtgaggaagatatatggcttcacgtagatgcagcctatgcgggagcagcatttttgtgtcctgaatacagacacctaatgaaaggtattgaacattctcaatcgttcgtaacgaatgcacataagtggctaccggttaatttcgattgctctgctatgtgggttaaaaatggttatgatataacgagagcattcgatgtacaaagaatttatttggatgatgtaaaaacgacaatcaagatcccagattacagacactggcaaatgccactaggccgtcgctttagagccttaaaattgtggacagttatgagaatttatggtgctgaaggtttgaaaacgcatatcagacagcaaatagaattagcacagtattttgcaaaactggtacgtgcggacgaacgttttgtgattgggcccgaaccgactatggcattggtctgttttagactgaaagacggtgacacaattacgcgacaattgttggaaaatataacgcaaaaaaagaaagtgtttatggtggccggaacgcacagggatagatacgtcattagattcgtgatctgctcccgattgactaagaaggaagacgtcgattacagctggagccaaataaagaaagaaaccgacctcatctattcagataaaatacacaataaagcacaaataccagctcttgaacaattcacttcaagagaactatgcgaaaaatctaagtaataa

SEQ ID NO: 3 - DNA, 1506 bp, Bombyx mori
atggacgcgaaccagtttcgggaattcggcagagcagtcatcgatatgttggcgagctacgctgaaaacataagggattatgatgttctgccgtctgttgaacctggttacttgttaagagctctgcctgaaagtgcacccgaacaaccggaggattggaaagacataatgaaagatttcaatcaatcaataatgccgggtgtaacacactggcaatctccgcagttccatgcatactttccttctggctcctcattcgcgagtattataggaaatatgcttagtgacggcttggcggttgtgggatttagttggatggctagccctgcgtgtacagaactggaggtggtcacgatgaattggcttggtaagctattagatttacctgaagaatttctaaattgctcttcgggtcccggaggcggagttattcaaggatcagcgagcgaagccaccttagttggattgcttgtagccaaagataaaactgttcgccgtttcatgaataataatccagatctcgatgaaaacgagataaaagcaaaacttgtagcttatacatccgaccaatgtaactcgtcagtagaaaaagccggcttacttggttctatgaaaatgaaactgttgaaagcggatgctgatggatgcctacgcggagaaacattgaaaagggcaatcgaagaagataagtcgcaaggacttataccctgctatgtcgtcgctaatctgggaacaactggaacttgtgcattcgatcctttacatgaattagggccaatatgtagtgaggaagatatatggcttcacgtagatgcagcctatgcgggagcagcatttttgtgtcctgaatacagacacctaatgaaaggtattgaacattctcaatcgttcgtaacgaatgcacataagtggctaccggttaatttcgattgctctgctatgtgggttaaaaatggttatgatataacgagagcattcgatgtacaaagaatttatttggatgatgtaaaaacgacaatcaagatcccagattacagacactggcaaatgccactaggccgtcgctttagagccttaaaattgtggacagttatgagaatttatggtgctgaaggtttgaaaacgcatatcagacagcaaatagaattagcacagtattttgcaaaactggtacgtgcggacgaacgttttgtgattgggcccgaaccgactatggcattggtctgttttagactgaaagacggtgacacaattacgcgacaattgttggaaaatataacgcaaaaaaagaaagtgtttatggtggccggaacgcacagggatagatacgtcattagattcgtgatctgctcccgattgactaagaaggaagacgtcgattacagctggagccaaataaagaaagaaaccgacctcatctattcagataaaatacacaataaagcacaaataccagctcttgaacaattcacttcaagagaactatgcgaaaaatctaagtaataa

SEQ ID NO: 4 - DNA, 1506 bp, Bombyx mori
atggacgcgaaccagtttcgggaattcggcagagcagtcatcgatatgttggcgagctacgctgaaaacataagggattatgatgttctgccgtctgttgaacctggttacttgttaagagctctgcctgaaagtgcacccgaacaaccggaggattggaaagacataatgaaagatttcaatcaatcaataatgccgggtgtaacacactggcaatctccgcagttccatgcatactttccttctggctcctcattcgcgagtattataggaaatatgcttagtgacggcttggcggttgtgggatttagttggatggctagccctgcgtgtacagaactggaggtggtcacgatgaattggcttggtaagctattagatttacctgaagaatttctaaattgctcttcgggtcccggaggcggagttattcaaggatcagcgagcgaagccaccttagttggattgcttgtagccaaagataaaactgttcgccgtttcatgaataataatccagatctcgatgaaaacgagataaaagcaaaacttgtagcttatacatccgaccaatgtcactcgtcagtagaaaaagccggcttacttggttctatgaaaatgaaactgttgaaagcggatgctgatggatgcctacgcggagaaacattgaaaagggcaatcgaagaagataagtcgcaaggacttataccctgctatgtcgtcgctaatctgggaacaactggaacttgtgcattcgatcctttacatgaattagggccaatatgtagtgaggaagatatatggcttcacgtagatgcagcctatgcgggagcagcatttttgtgtcctgaatacagacacctaatgaaaggtattgaacattctcaatcgttcgtaacgaatgcacataagtggctaccggttaatttcgattgctctgctatgtgggttaaaaatggttatgatataacgagagcattcgatgtacaaagaatttatttggatgatgtaaaaacgacaatcaagatcccagattacagacactggcaaatgccactaggccgtcgctttagagccttaaaattgtggacagttatgagaatttatggtgctgaaggtttgaaaacgcatatcagacagcaaatagaattagcacagtattttgcaaaactggtacgtgcggacgaacgttttgtgattgggcccgaaccgactatggcattggtctgttttagactgaaagacggtgacacaattacgcgacaattgttggaaaatataacgcaaaaaaagaaagtgtttatggtggccggaacgcacagggatagatacgtcattagattcgtgatctgctcccgattgactaagaaggaagacgtcgattacagctggagccaaataaagaaagaaaccgacctcatctattcagataaaatacacaataaagcacaaataccagctcttgaacaattcacttcaagagaactatgcgaaaaatctaagtaataa

SEQ ID NO: 5 - protein, 500 aa, Bombyx mori
MDANQFREFGRAVIDMLASYAENIRDYDVLPSVEPGYLLRALPESAPEQPEDWKDIMKDFNQSIMPGVTHWQSPQFHAFYPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCNSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 6 - protein, 500 aa, Bombyx mori
MDANQFREFGRAVIDMLASYAENIRDYDVLPSVEPGYLLRALPESAPEQPEDWKDIMKDFNQSIMPGVTHWQSPQFHAFYPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCHSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 7 - protein, 500 aa, Bombyx mori
MDANQFREFGRAVIDMLASYAENIRDYDVLPSVEPGYLLRALPESAPEQPEDWKDIMKDFNQSIMPGVTHWQSPQFHAYFPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCNSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 8 - protein, 500 aa, Bombyx mori
MDANQFREFGRAVIDMLASYAENIRDYDVLPSVEPGYLLRALPESAPEQPEDWKDIMKDFNQSIMPGVTHWQSPQFHAYFPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCHSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 9 - protein, 608 aa, Bombyx mori
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMDANQFREFGRAVIDMLASYAENIRDYDVLPSVEPGYLLRALPESAPEQPEDWKDIMKDFNQSIMPGVTHWQSPQFHAFYPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCNSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 10 - protein, 608 aa, Bombyx mori
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMDANQFREFGRAVIDMLASYAENIRDYDVLPSVEPGYLLRALPESAPEQPEDWKDIMKDFNQSIMPGVTHWQSPQFHAFYPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCHSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 11 - protein, 608 aa, Bombyx mori
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMDANQFREFGRAVIDMLASYAENIRDYDVLPSVEPGYLLRALPESAPEQPEDWKDIMKDFNQSIMPGVTHWQSPQFHAYFPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCNSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 12 - protein, 608 aa, Bombyx mori
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMDANQFREFGRAVIDMLASYAENIRDYDVLPSVEPGYLLRALPESAPEQPEDWKDIMKDFNQSIMPGVTHWQSPQFHAYFPSGSSFASIIGNMLSDGLAVVGFSWMASPACTELEVVTMNWLGKLLDLPEEFLNCSSGPGGGVIQGSASEATLVGLLVAKDKTVRRFMNNNPDLDENEIKAKLVAYTSDQCHSSVEKAGLLGSMKMKLLKADADGCLRGETLKRAIEEDKSQGLIPCYVVANLGTTGTCAFDPLHELGPICSEEDIWLHVDAAYAGAAFLCPEYRHLMKGIEHSQSFVTNAHKWLPVNFDCSAMWVKNGYDITRAFDVQRIYLDDVKTTIKIPDYRHWQMPLGRRFRALKLWTVMRIYGAEGLKTHIRQQIELAQYFAKLVRADERFVIGPEPTMALVCFRLKDGDTITRQLLENITQKKKVFMVAGTHRDRYVIRFVICSRLTKKEDVDYSWSQIKKETDLIYSDKIHNKAQIPALEQFTSRELCEKSK

SEQ ID NO: 13 - DNA, 7422 bp, Artificial Sequence
  6OMT-4′OMT-CNMT_pACYC184
aactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgcttcgttaatacagatgtaggtgttccacagggtagccagcagcatcctgcgatgcagatccggaacataatggtgcagggcgcttgtttcggcgtgggtatggtggcaggccccgtggccgggggactgttgggcgctgccggcacctgtcctacgagttgcatgataaagaagacagtcataagtgcggcgacgatagtcatgccccgcgcccaccggaaggagctaccggacagcggtgcggactgttgtaactcagaataagaaatgaggccgctcatggcgttgactctcagtcatagtatcgtggtatcaccggttggttccactctctgttgcgggcaacttcagcagcacgtaggggacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtaccgatcccgcgaaattaatacgactcactataggggaattgtgagcggataacaattcccctctagaaataattttgtttaactttaagaaggagatataccatgttagtgaagaagaaggacaatctctcatctcaagctaaactgtggaacttcatttatggttttgctgaatcactagtcctcaaatgtgcagtgcaacttgatctagccaacataattcacaacagtggcacgtccatgactctttccgagttatcttcgcgtcttccaagtcaacctgtcaatgaagacgccttgtatcgagtcatgcgttacttggttcacatgaagctattcacaaaagcatcaatagatggagaactaagatatggacttgcaccaccagctaagtatcttgttaaaggttgggataaatgtatggttggctcaattttagcaatcactgataaagatttcatggcaccatggcattaccttaaggatggattatcaggcgaaagtggtacagcgtttgagaaggccttggggacgaatatatgggggtacatggcagagcaccctgagaaaaaccagctatttaatgaagcaatggctaatgattcaaggcttattatgtctgcattggtgaaagaatgtggaaatatttttaatggtataactacacttgtggatgttggtggtggtactggaactgctgtgaggaatattgccaatgcatttccacatataaagtgtactgtttatgatcttcctcatgtcattgctgattctcctgggtactccgaagttcattgcgtggcaggtgatatgttcaagttcataccaaaggctgatgctatcatgatgaagtgcatccttcacgactgggatgacaaagaatgcattgaaattctaaagcgatgcaaggaggcagtaccagtcaaaggcgggaaagtgattatagtcgacattgtcttaaatgtgcaatcagaacatccttataccaagatgagactgactttggatttggacatgatgctcaacactggaggaaaagagaggactgaagaggaatggaagaagctcatccatgatgcagggtacaaagggcataagataacacaaattactgctgtacaatctgtgattgaggcttatccatattagatctcgatcccgcgaaattaatacgactcactataggggaattgtgagcggataacaattcccctctagaaataattttgtttaactttaagaaggagatataccatgtctttccatgggaaagatgatgttctggacatcaaagctcaagctcatgtgtggaaaatcatctatggttttgcagattccctagtcctccgatgtgcagtggaacttggaatcgtcgacatcattgataacaacaaccaacccatggcacttgccgatctggcatctaagcttcctgtttccgatgtgaattgcgataatttgtatcggatattacgatacttggtgaaaatggaaatactgagagtggaaaaatctgatgatggtcagaagaagtacgcgcttgaacctattgcaacattgctttcaaggaatgcgaagaggagtatggttccaatgattcttggaatgactcaaaaagattttatgactccttggcattcaatgaaggatggcttaagtgacaatggtactgcttttgagaaggccatgggaatgactatatgggagtacttggaaggacaccctgatcaaagccaattattcaatgaaggcatggccggtgaaacaaggcttctcacttcttcactcatatctggaagtagagatatgtttcaaggtattgactcacttgttgatgttggtggaggaaatggtactactgtcaaggccatttctgacgcatttccacatatcaagtgcaccctctttgatctccctcatgtcattgccaattcctatgaccttcctaatattgaacgaattggtggcgacatgtttaaatccgtgcccagtgcccaagctatcatactcaagctaattttgcacgattggaatgacgaagactcgatcaagattttaaagcaatgcagaaatgcagtgccaaaagatggaggaaaagtgattatagtggatgtggcattagatgaggagtcagaccatgagcttagcagcacacgattgatccttgatatcgatatgttggtgaacactggtggtaaagagcggactaaagaggtttgggagaaaattgtgaaaagtgcaggatttagtggttgcaaaatcaggcacatagcggctatacaatcagtcattgaggtttttccataggatccgatcccgcgaaattaatacgactcactataggggaattgtgagcggataacaattcccctctagaaataattttgtttaactttaagaaggagatataccatggctgtggaagcaaagcaaacaaagaaggcagccatagtagagttgttaaaacagttggagctgggcttggttccatatgatgatattaagcagctcataaggagggaactggcaaggcgcctgcaatggggttataaacctacttatgaagaacaaatagctgaaatccaaaacttaactcattctctgcgacaaatgaaaattgcaacagaggttgagaccttggattcacaattgtacgagattcctattgagtttctaaagattatgaatggaagtaacttaaaaggaagttgttgctacttcaaagaagattcaacaacattagatgaagctgagatagcgatgctggatttatactgcgagagagctcaaatccaagatggacagagtgttcttgatcttggatgtgggcaaggagctcttacattacatgttgcacagaaatataaaaactgtcgcgtaacagcagtaacaaattcagtttcacaaaaagagtacattgaagaagaatcaaggagacgtaatttgttgaatgtggaagtcaaattggcagacataaccacacatgagatggctgagacatacgatcgtattttggtaatagagttgtttgagcacatgaagaactatgaacttctcctgaggaaaatctcagagtggatatcgaaagatgggcttctctttctagagcacatatgccacaagacctttgcttaccactatgagcctctagacgacgacgattggtttacagagtacgtgtttcctgctgggactatgatcataccatctgcatcgttctttttgtatttccaggatgacgtttcggttgtgaaccattggactcttagtgggaagcacttttcgcgtaccaatgaggaatggttgaagagattggacgcaaaccttgatgttattaaaccaatgtttgagactttaatgggaaatgaggaagaggcagtgaagttgattaactattggagaggattttgtttatctggaatggaaatgtttggatataacaatggtgaagaatggatggcaagtcatgttctgttcaagaaaaaatgagaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgcctgcaggcatgcaagcttggcactggccgtcgttttacaacgtcgtgactgggaaaaccctggcgttacccaacttaatcgccttgcagcacatccccctttcgccagatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttcc

SEQ ID NO: 14 - DNA, 5903 bp, Artificial Sequence
  DHPAAS-pTrcHis2B
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatggacgcgaaccagtttcgggaattcggcagagcagtcatcgatatgttggcgagctacgctgaaaacataagggattatgatgttctgccgtctgttgaacctggttacttgttaagagctctgcctgaaagtgcacccgaacaaccggaggattggaaagacataatgaaagatttcaatcaatcaataatgccgggtgtaacacactggcaatctccgcagttccatgcatactttccttctggctcctcattcgcgagtattataggaaatatgcttagtgacggcttggcggttgtgggatttagttggatggctagccctgcgtgtacagaactggaggtggtcacgatgaattggcttggtaagctattagatttacctgaagaatttctaaattgctcttcgggtcccggaggcggagttattcaaggatcagcgagcgaagccaccttagttggattgcttgtagccaaagataaaactgttcgccgtttcatgaataataatccagatctcgatgaaaacgagataaaagcaaaacttgtagcttatacatccgaccaatgtcactcgtcagtagaaaaagccggcttacttggttctatgaaaatgaaactgttgaaagcggatgctgatggatgcctacgcggagaaacattgaaaagggcaatcgaagaagataagtcgcaaggacttataccctgctatgtcgtcgctaatctgggaacaactggaacttgtgcattcgatcctttacatgaattagggccaatatgtagtgaggaagatatatggcttcacgtagatgcagcctatgcgggagcagcatttttgtgtcctgaatacagacacctaatgaaaggtattgaacattctcaatcgttcgtaacgaatgcacataagtggctaccggttaatttcgattgctctgctatgtgggttaaaaatggttatgatataacgagagcattcgatgtacaaagaatttatttggatgatgtaaaaacgacaatcaagatcccagattacagacactggcaaatgccactaggccgtcgctttagagccttaaaattgtggacagttatgagaatttatggtgctgaaggtttgaaaacgcatatcagacagcaaatagaattagcacagtattttgcaaaactggtacgtgcggacgaacgttttgtgattgggcccgaaccgactatggcattggtctgttttagactgaaagacggtgacacaattacgcgacaattgttggaaaatataacgcaaaaaaagaaagtgtttatggtggccggaacgcacagggatagatacgtcattagattcgtgatctgctcccgattgactaagaaggaagacgtcgattacagctggagccaaataaagaaagaaaccgacctcatctattcagataaaatacacaataaagcacaaataccagctcttgaacaattcacttcaagagaactatgcgaaaaatctaagtaataactcgagatctgcagctggtaccatatgggaattcgaagctttctagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 15 - protein, 518 aa, Papaver somniferum
MGSLPANNFESMSLCSQNPLDPDEFRRQGHMIIDFLADYYKNVEKYPVRTQVDPGYLKKRLPESAPYNPESIETILEDVTNDIIPGLTHWQSPNYFAYFPSSGSIAGFLGEMLSTGFNVVGFNWMSSPAATELESIVMNWLGQMLTLPKSFLFSSDGSSGGGGVLQGTTCEAILCTLTAARDKMLNKIGRENINKLVVYASDQTLSALQKAAQIAGINPKNFLAIATSKATNFGLSPNSLQSTILADIESGLVPLFLCATVGTTSSTAVDPIGPLCAVAKLHGIWVHIDAAYAGSACICPEFRHFIDGVEDADSFSLNAHKWFFTTLDCCCLWVKDSDSLVKALSTSPEYLKNKATDSKQVIDYKDWQIALSRRFRSMKLWLVLRSYGIANLRTFLRSHVKMAKHFQGLIGMDNRFEIVVPRTFAMVCFRLKPAAIFRKKIVEDDHIEAQTNEVNAKLLESVNASGKIYMTHAVVGGVYMIRFAVGATLTEERHVTGAWKVVQEHTDAILGALGEDVC

SEQ ID NO: 16 - DNA, 1554 bp, Papaver somniferum
atgggaagtcttccagctaataactttgaaagcatgtcgctgtgttcgcaaaatccacttgatccagatgaattcagaaggcaaggtcacatgattattgatttccttgctgattactacaaaaatgttgagaaatatccagttagaacccaagtcgatcccggttatttgaagaaaaggttacccgaatcagctccgtacaatcctgaatccattgaaaccattcttgaagatgtgacaaatgatatcatccctggtctaactcactggcaaagtccaaattactttgctttttatccttctagtggttctatcgctggtttcctaggggaaatgctaagtaccggatttaatgttgtcgggtttaattggatgtcatctccggccgcaactgagttggagagtattgttatgaattggcttggccagatgcttacgcttcccaaatcatttctcttttcatcagacggaagttcgggaggtggaggagttttgcaagggactacttgtgaagccattttatgtactctaactgcggcaagagataaaatgctgaacaaaattggtagagaaaatattaacaagttggttgtttatgcttctgatcaaaccctaagtgcactacagaaagctgctcaaattgctgggattaatcctaagaatttccttgctatcgcaacctccaaggctacaaattttggtctctctccaaattcacttcaatcgacaattcttgctgatatcgaatccgggttagttccattgtttctctgtgccactgtcggaacaacttcttcaacagccgtagatcctattggcccactttgcgcggtggcaaaattgcacggtatttgggttcacattgatgctgcatacgctggaagtgcatgtatctgcccagagttcaggcacttcatcgatggtgtggaagatgcagactcatttagtctaaatgcacacaagtggttctttactactttggattgttgctgtttatgggtgaaagactctgattcactggtcaaggcattatcaacaagtccagaatatttgaagaacaaagcaactgattccaaacaagttatcgattacaaagattggcaaatagcgctcagcagaagattccgatccatgaaactctggttagtacttcgcagctatggaattgctaacttaagaaccttccttaggagccatgttaaaatggctaagcactttcaggggctcattggtatggacaacaggtttgagattgtagttcctagaacatttgccatggtgtgctttcgccttaaaccagctgccatttttaggaaaaaaatagttgaagatgatcacattgaagctcaaacaaatgaggtaaatgcgaaattgcttgaatcagtcaatgcgtccgggaagatatacatgactcatgctgttgttggaggggtgtacatgattcggtttgccgtcggggcaacactgacagaggaaagacatgtcactggggcttggaaggtggtacaggagcatacagatgccatacttggtgcactaggtgaggatgtttgt

SEQ ID NO: 17 - DNA, 41 bp, Artificial Sequence
  forward primer
ctttgctttttatccttctagtggttctatcgctggtttcc

SEQ ID NO: 18 - DNA, 41 bp, Artificial Sequence
  reverse primer
ggaaaccagcgatagaaccactagaaggataaaaagcaaag

SEQ ID NO: 19 - DNA, 40 bp, Artificial Sequence
  forward primer
gcttctgatcaaaccaacagtgcactacagaaagctgctc

SEQ ID NO: 20 - DNA, 40 bp, Artificial Sequence
  reverse primer
gagcagctttctgtagtgcactgttggtttgatcagaagc

SEQ ID NO: 21 - protein, 531 aa, Papaver somniferum
MGSLNTEDVLEHSSAFGATNPLDPEEFRRQGHMIIDFLADYYRDVEKYPVRSQVEPGYLRKRLPETAPYNPESIETILQDVTSEIIPGLTHWQSPNYYAYFPSSGSVAGFLGEMLSTGFNVVGFNWMSSPAATELEGIVMDWFGKMLNLPKSYLFSGTGGGVLQGTTCEAILCTLTAARDRKLNKIGREHIGRLVVYGSDQTHCALQKAAQIAGINPKNFRAVKTFKANSFGLAASTLREVILEDIEAGLIPLFVCPTVGTTSSTAVDPIGPICEVAKEYEMWVHIDAAYAGSACICPEFRHFIDGVEEADSFSLNAHKWFFTTLDCCCLWVKDPSSLVKALSTNPEYLRNKATESRQVVDYKDWQIALIRRFRSMKLWMVLRSYGVTNLRNFLRSHVRMAKTFEGLVGADRRFEITVPRTFAMVCFRLLPPTTVKVCGENGVHQNGNGVIAVLRNENEELVLANKLNQVYLRQVKATGSVYMTHAVVGGVYMIRFAVGSTLTEERHVIHAWEVLQEHADLILSKFDEANF

SEQ ID NO: 22 - DNA, 1593 bp, Papaver somniferum
atgggtagtcttaacacagaagatgttcttgaacacagttcagctttcggtgcaacaaacccattagacccagaagaattcagaagacaaggtcacatgataatcgacttcttagctgattattacagagatgtcgagaaatatccagttcgaagtcaagtagaacccggttatctacgtaaaagattaccagaaacagctccatacaatccagaatctatcgaaacgattcttcaagatgtgacgagtgagattattccagggttaacacattggcaaagtcctaattactatgcttatttcccttccagtggttccgttgctggattcctcggtgaaatgcttagtactggttttaatgtcgttggttttaactggatgtcttcacctgctgctacagaactcgagggtattgttatggattggttcggcaaaatgcttaaccttccaaaatcatacttgttctctggtaccggtggtggagttttacagggaactacttgtgaagctatcttatgtacattaacagctgcaagagacagaaagttgaacaaaatcggtcgtgaacatatcggaagattagttgtttatggatctgatcagactcactgtgcactacagaaagctgctcagattgcaggaatcaaccccaagaacttccgtgctgttaagacgtttaaagctaattcattcggattagcagcttcaactctaagagaagttattcttgaagatattgaagccgggttgatccctctgtttgtatgtccaacggtcggaactacatcatcgactgcagtggatccaatcggtcctatctgtgaagtggcgaaagaatacgaaatgtgggttcacatcgacgcagcttacgctggaagtgcatgtatctgtcccgagtttagacactttatcgacggagtggaggaagcagattcattcagtctcaatgcgcataaatggtttttcacaactttggattgttgttgtctttgggttaaagatccaagttccctggttaaagctctttccacaaatcctgagtacttgagaaacaaagctacagagtcaagacaggtcgttgactacaaagactggcagatcgcactcattcgccgattccgatccatgaagctttggatggttttacgtagctatggtgtgactaatctgagaaatttcttgaggagtcatgttagaatggcaaagacatttgagggtctcgttggtgcggataggagattcgaaattactgtgcctaggacgtttgctatggtctgcttccgccttttacccccaacaaccgtaaaggtatgcggtgaaaatggagtacaccagaatggaaacggggtcattgcagtactacgcaatgaaaatgaagaattagtccttgctaataagctgaatcaagtgtatttgagacaggtcaaggcaacaggtagtgtttatatgacacatgcggtcgtcggaggtgtctacatgattcggttcgcagtcggttcgaccttgacagaggaacgccatgttattcatgcttgggaggttttgcaagagcatgcagatctgattcttagtaagttcgatgaagcaaatttt

SEQ ID NO: 23 - DNA, 40 bp, Artificial Sequence
  forward primer
gtcctaattactatgctttttacccttccagtggttccgt

SEQ ID NO: 24 - DNA, 40 bp, Artificial Sequence
  reverse primer
acggaaccactggaagggtaaaaagcatagtaattaggac

SEQ ID NO: 25 - DNA, 40 bp, Artificial Sequence
  forward primer
tatggatctgatcagactaactgtgcactacagaaagctg

SEQ ID NO: 26 - DNA, 40 bp, Artificial Sequence
  reverse primer
cagctttctgtagtgcacagttagtctgatcagatccata

SEQ ID NO: 27 - DNA, 576 bp, thalictrum flavum
atgaaattaatcctgaccggtcgcccgtttttacatcatcagggcatcatcaaccaggtgagcaccgtcaccaaagtcattcaccacgaactggaagtggcggccagcgccgatgatatctggacggtttacagctggccgggtctggcgaaacatctgccggatctgctgcctggcgcgtttgaaaagctggaaattatcggcgatggcggcgttggcaccatcctcgatatgacctttgttccgggcgaattcccgcatgaatacaaagaaaaatttattctggttgataacgaacatcgcctgaaaaaagtgcagatgattgaaggcggttatctggatctcggcgtgacctattatatggataccattcatgtggtgccgacgggtaaagatagctgcgtgattaaatcgtccaccgaatatcacgttaaaccggaatttgtgaaaatcgttgaaccgctgatcaccaccggcccgctggcagcgatggcggatgccatcagcaaactggtgctggagcataaaagtaaaagcaacagcgatgaaatcgaagcggcgattattaccgtttaa

SEQ ID NO: 28 - protein, 191 aa, Thalictrum flavum
MKLILTGRPFLHHQGIINQVSTVTKVIHHELEVAASADDIWTVYSWPGLAKHLPDLLPGAFEKLEIIGDGGVGTILDMTFVPGEFPHEYKEKFILVDNEHRLKKVQMIEGGYLDLGVTYYMDTIHVVPTGKDSCVIKSSTEYHVKPEFVKIVEPLITTGPLAAMADAISKLVLEHKSKSNSDEIEAAIITV

SEQ ID NO: 29 - protein, 641 aa, Papaver somniferum
MRKVIKYDMEVAVSADSVWAVYSSPDIPRLLRDVLLPGVFEKLDVIEGNGGVGTVLDIVFPPGAVPRSYKEKFVNIDREKRLKEVIMIEGGYLDMGCTFYLDRIHVVEKTKSSCVIESSIVYDAKEECADAMSKLITTEPLKSMAEVISNYVIQKESFSARNILSKQSVVKKEIRYDLEVPISADSIWSVYSCPDIPRLLRDVLLPGVFEKLDVIEGDGGVGTVLDIVFPPGAVPRSYKEKFVNIDREKRLKEVIMIEGGYLDMGCTFYLDRIHVVEKSLSSCVIESSIVYEVKEEYVDAMSKLITTEPLKSMAEVISNYVIQRESFSARNILNKNSLVKKEIRYDLEVPTSADSIWSVYSCPDIPRLLRDVLLPGVFQKLDVIEGNGGVGTVLDIVFPPGAVPRSYKEKFVNINHEKRLKEVIMIEGGYLDMGCTSYLDRIHVVEKTSKSCIIKSSVVYEVKQECVEAMSKLITTEPLKSMAEVISNYAMKQQSVSERNIPKKQSLLRKEITYETEVQTSADSIWNVYSSPDIPRLLRDVLLPGVFEKLDVIAGNGGVGTVLDIAFPLGAVRRRYKEKFVKINHEKRLKEVVMIEGGYLDMGCTFYMDRIHVFEKTPNSCVIESSIITKLKKSMLVKWLS

SEQ ID NO: 30 - DNA, 1926 bp, Papaver somniferum
atgcgcaaagtgattaaatacgatatggaagtggcggtttctgctgacagtgtctgggcggtctattcatcgccagatattccacgcttgttgcgtgacgtcttactgcccggcgtgtttgaaaaattagacgtgatcgaaggcaatggcggcgtcggcaccgtgctggatattgttttcccgccaggcgcggttccgcgttcttataaagagaagttcgtgaatattgatcgcgaaaaacgcctgaaagaggtcattatgattgaagggggttatcttgatatgggctgcaccttctatctggaccgtattcacgttgtcgaaaaaaccaaaagcagttgcgtcattgaatccagtattgtgtacgacgcgaaagaagaatgcgccgatgcgatgagtaaactgattactaccgagccgttgaaatcgatggcagaagtgattagcaattatgtcattcaaaaagagagttttagcgcgcgtaatattctgtcaaaacaaagcgtggtaaaaaaggaaattcgctatgacctggaggtgccgatttctgcggattctatctggagcgtatatagctgtccggacatcccgcgtctgttacgtgatgtgctgttgcctggcgtgttcgaaaagctggatgtgatcgagggcgatggcggcgttggtaccgttctggacattgtcttcccgccgggggcggtgccgcgcagctacaaagagaagtttgtcaatatcgaccgcgagaaacgtctcaaagaagttatcatgatcgaaggtggctacctggatatgggctgtaccttttacctcgaccgcattcatgtggttgaaaaatctctgtcttcctgcgtgattgagagctccatcgtttatgaagtgaaagaagagtatgttgatgccatgtcgaaactgatcaccacggaaccgctgaaaagcatggctgaagttatcagcaactatgtgattcagcgtgaaagcttcagcgcccgcaatatcctgaataaaaatagcctggttaagaaagagattcgttatgatctggaagtcccgacctctgccgacagcatctggtcggtttacagctgcccggatatcccacgtttattacgcgacgttttgctgccgggcgtattccagaaactcgatgttatagaaggcaacggcggtgtcggtacggtactggatatcgtctttccaccgggtgcggtaccgcgcagttataaagagaaattcgttaacatcaaccatgaaaagcgtctgaaggaggttattatgattgaaggcggttaccttgatatggggtgtaccagctatctcgatcgcatccatgtcgttgagaaaacaagcaaatcctgtattattaagagcagcgttgtttatgaggtgaagcaggaatgtgtggaagcgatgagcaaattgattaccaccgaaccactgaaatcaatggcggaagtcatcagtaactacgcaatgaaacagcagagcgtcagcgaacgtaacattccgaaaaaacagtcgctgctgcgtaaagaaatcacctacgaaaccgaagtgcagaccagtgctgatagcatttggaacgtgtattccagcccggatattcctcgcctgctgcgcgatgtcctgcttcccggtgtttttgaaaaactggatgtaattgccggtaacggtggtgtgggcacggtgttggatatcgcctttccgctgggcgcagtgcgtcgccgctacaaagaaaaatttgttaaaattaaccacgaaaagcgcttaaaagaagtggtgatgatcgaaggtggctatctggacatgggttgtacgttttatatggatcgtatccacgtatttgagaaaacgccgaacagctgcgttattgaaagttcgattatcactaaacttaaaaaaagtatgctggtgaaatggctgagttaa

(19) United States
(12) Patent Application Publication
(10) Pub. No.: US 2021/0395717 A1
(43) Pub. Date: Dec. 23, 2021

(54) RECOMBINANT HOST CELL FOR PRODUCING BENZYLISOQUINOLINE ALKALOID (BIA) AND NOVEL METHOD FOR PRODUCING BENZYLISOQUINOLINE ALKALOID (BIA)

(71) Applicant: NATIONAL UNIVERSITY CORPORATION KOBE UNIVERSITY, Kobe-shi, Hyogo (JP)

(72) Inventors: Christopher John VAVRICKA JR., Kobe-shi, Hyogo (JP); Tomohisa HASUNUMA, Kobe-shi, Hyogo (JP); Michihiro ARAKI, Kobe-shi, Hyogo (JP); Akihiko KONDO, Kobe-shi, Hyogo (JP)

(73) Assignee: NATIONAL UNIVERSITY CORPORATION KOBE UNIVERSITY, Kobe-shi, Hyogo (JP)

(21) Appl. No.: 17/290,120

(22) PCT Filed: Oct. 30, 2019
(86) PCT No.: PCT/JP2019/042694

§ 371 Date: Apr. 29, 2021

(30) Foreign Application Priority Data
Oct. 30, 2018 (JP) 2018-203904

(51) Int. Cl. C12N 9/88; C12N 9/10; C12P 17/12

(52) U.S. Cl. CPC C12N 9/88; C12Y 401/01025; C12Y 401/01028; C12Y 402/01078; C12P 17/12; C12N 9/1007; C12Y 201/01116; C12Y 201/0114; C12Y 201/01128

(57) ABSTRACT

The purpose of the present invention is to provide a recombinant host cell which is capable of efficiently and easily producing a benzylisoquinoline alkaloid (BIA), in particular, tetrahydropapaveroline, 3-hydroxycoclaurine, 3-hydroxy-N-methylcoclaurine and/or reticuline, and a method for efficiently and easily producing these BIAs using the host cell. The present invention pertains to a recombinant host cell for producing a benzylisoquinoline alkaloid (BIA), in particular, tetrahydropapaveroline (THP), 3-hydroxycoclaurine, 3-hydroxy-N-methylcoclaurine and/or reticuline, in which a wild-type aromatic aldehyde synthase (AAS) or a mutant thereof and a wild-type aromatic amino acid decarboxylase (AAAD) or a mutant thereof are expressed.

### TECHNICAL FIELD

The present invention relates to a recombinant host cell for producing a benzylisoquinoline alkaloid (BIA), and a novel method for producing a benzylisoquinoline alkaloid (BIA).

### BACKGROUND ART

Benzylisoquinoline alkaloid (BIA) derivatives are a diverse group of compounds including useful medicaments, e.g., analgesics such as morphine and codeine, and antimicrobials such as berberine. Many of these benzylisoquinoline alkaloid derivatives are synthesized from tyrosine via benzylisoquinoline alkaloids (BIAs) such as tetrahydropapaveroline (THP), norcoclaurine and reticuline in various plants. Tetrahydropapaveroline (THP), norcoclaurine and reticuline are therefore important intermediates in the biosynthetic pathways of many benzyl quinoline alkaloid derivatives. Such tetrahydropapaveroline (THP), norcoclaurine, reticuline are not used as-is for the treatment of diseases, but are used industrially as pharmaceutical raw materials to produce oxycodone, oxymorphone, nalbuphine, naloxone, naltrexone, buprenorphine, etorphine, and the like.

Conventionally, production of benzylisoquinoline alkaloids (BIAs) and derivatives thereof are mostly relied on extraction from plants. Several benzylisoquinoline alkaloids (BIAs) have been chemically synthesized by total synthesis (see Non Patent Document 1). However, from the viewpoint of production stability and efficiency, the development of other production methods has been required. For example, bioproduction with microorganisms draws attention because its products do not include other plant metabolites, and thus can efficiently produce required benzylisoquinoline alkaloids (BIAs) (see Non Patent Documents 2 to 4). However, the yields are less than 10 mg per liter. Thus, further optimization of the bioproduction method is required in order to meet industrial demands.

### PRIOR ART DOCUMENTS

### Non Patent Documents

Non Patent Document 1: Gates M. et al, The synthesis of morphine, J Am Chem Soc 74, 1109-1110 (1952)
Non Patent Document 2: Galanie, S., Thodey, K., Trenchard, I. J., Filsinger Interrante, M. &Smolke, C. D. Complete biosynthesis of opioids in yeast, Science 349, 1095-1100 (2015)
Non Patent Document 3: Nakagawa, A. et al. (R,S)-Tetrahydropapaveroline production by stepwise fermentation using engineered Escherichia coli. Sci. Rep. 4, 6695 (2014)
Non Patent Document 4: Nakagawa, A. et al. Total biosynthesis of opiates by stepwise fermentation using engineered Escherichia coli. Nat. Commun. 7, 10390 (2016)

### SUMMARY OF INVENTION

### Technical Problem

In view of the status above, an object of the present invention is to provide a microorganism capable of efficiently producing a benzylisoquinoline alkaloid (BIA) and a method for producing a benzylisoquinoline alkaloid (BIA) using the same. Specifically, an object of the present invention is to provide a recombinant host cell capable of efficiently and easily producing a benzylisoquinoline alkaloid (BIA) such as tetrahydropapaveroline (THP), norcoclaurine, reticuline, which is an intermediate of a biosynthetic pathway of a number of benzylisoquinoline alkaloid (BIA) derivatives, and to provide a method for efficiently and easily producing a benzylisoquinoline alkaloid (BIA) such as tetrahydropapaveroline (THP), norcoclaurine, reticuline, or the like using the host cell.

### Solution to Problem

To solve the above problems, the present inventors have applied a synthetic biology-based approach in a method for producing a benzylisoquinoline alkaloid (BIA) such as tetrahydropapaveroline (THP), norcoclaurine, reticuline using a microorganism to design a novel biosynthetic pathway, and have successfully identified a bifunctional enzyme, an aromatic aldehyde synthase (AAS). The present inventors have also found that, by introducing mutations to change specific residues of aromatic amino acid decarboxylases (AAADs), such as tyrosine decarboxylase (TyDC) or dopa decarboxylase (DDC), the resulting enzymes can exhibit 4-hydroxyphenylacetaldehdye synthase (4-HPAAS) and 3,4-dihydroxyphenylacetaldehyde synthase (DHPAAS) activities. Thus, the summary of the present invention is as shown below.

[1] A recombinant host cell for producing a benzylisoquinoline alkaloid (BIA), expressing a wild-type or a variant of an aromatic aldehyde synthase (AAS) and an aromatic amino acid decarboxylase (AAAD) of a heterologous species.

[2] The recombinant host cell according to [1], wherein the benzylisoquinoline alkaloid (BIA) is tetrahydropapaveroline (THP), norcoclaurine, 3-hydroxycoclaurine, 3-hydroxy-N-methylcoclaurine and/or reticuline.

[3] The recombinant host cell according to [1] or [2], wherein the heterologous species is an insect, a plant, or a microorganism.

[4] The recombinant host cell according to [3], wherein the heterologous species is an insect selected from the group consisting of Bombyx mori, Camponotus floridanus, Apis mellifera, Aedes aegypti, and Drosophila melanogaster, Papaver somniferum, or Pseudomonas putida.

[5] The recombinant host cell according to any one of [1] to [4], wherein the host cell is E. coli.

[6] The recombinant host cell according to any one of [1] to [5], wherein the aromatic aldehyde synthase (AAS) is 3,4-dihydroxyphenylacetaldehyde synthase (DHPAAS), 4-hydroxyphenylacetaldehyde synthase (4-HPAAS).

[7] The recombinant host cell according to [6], wherein the aromatic aldehyde synthase (AAS) is derived from an insect and a mutation in the variant of the aromatic aldehyde synthase (AAS) is at least one selected from the group consisting of Asn192His, Phe79Tyr, and Tyr80Phe.

[8] The recombinant host cell according to [6], wherein the aromatic amino acid decarboxylase (AAAD) is tyrosine decarboxylase (TyDC) derived from a plant, and a mutation in the variant of the tyrosine decarboxylase (TyDC) is at least one selected from the group consisting of Leu205Asn, Phe99Tyr, and Tyr98Phe, or at least one selected from the group consisting of His203Asn, Phe101Tyr, and Tyr100Phe.

[9] The recombinant host cell according to [6], wherein the aromatic amino acid decarboxylase (AAAD) is DOPA decarboxylase (DDC) derived from a microorganism, and a mutation in the variant of the DOPA decarboxylase (DDC) is at least one selected from the group consisting of Tyr79Phe, Phe80Tyr, and His181Asn.

[10] The recombinant host cell according to any one of [1] to [9], further expressing norcoclaurine synthase (NCS).

[11] The recombinant host cell according to any one of [1] to [10], further expressing at least one enzyme selected from the group consisting of norcoclaurine 6-O-methyltransferase (6′OMT), 3′-hydroxy-N-methyl-(S)-coclaurine-4′-O-methyltransferase (4′OMT), coclaurine-N-methyltransferase (CNMT), and N-methylcoclaurine 3-hydroxylase.

[12] A method for producing a benzylisoquinoline alkaloid (BIA), comprising a step of culturing the recombinant host cell according to any one of [1] to [11] in a L-DOPA or tyrosine-containing culture medium.

[13] A method for producing a benzylisoquinoline alkaloid (BIA), comprising a step of causing a wild-type or a variant of an aromatic aldehyde synthase (AAS), an aromatic amino acid decarboxylase (AAAD) to act on L-DOPA or tyrosine in a cell-free system.

[14] The production method according to [13], wherein the wild-type or the variant of the aromatic aldehyde synthase (AAS), the aromatic amino acid decarboxylase (AAAD) is an enzyme obtained from the recombinant host cell according to any one of [1] to [11].

### Advantageous Effects of Invention

According to the present invention, by using a recombinant host cell expressing an aromatic aldehyde synthase (AAS) that is a bifunctional enzyme, or the like, a benzylisoquinoline alkaloid (BIA) such as tetrahydropapaveroline (THP), norcoclaurine, 3-hydroxycoclaurine, 3-hydroxy-N-methylcoclaurine, reticuline can be efficiently and easily produced.

### BRIEF DESCRIPTION OF DRAWINGS

FIG. 1 is a diagram showing THP synthesis pathways for reticuline production found in M-Path searches.

FIG. 2 is a diagram showing predicted yields of THP in symmetric DDC-DHPAAS pathways and MAO-mediated asymmetric pathways.

FIG. 3 is a diagram showing structural analysis of AAAD and 4-HPAAS and DHPAAS. The left shows the structure of D. melanogaster-derived DDC complexed with PLP (internal aldimine), the middle shows the structure models of P. somniferum TyDC1 complexed with PLP-4-HPAA, and the right shows the structure models of B. mori-derived DHPAAS complexed with PLP-DOPA.

FIG. 4 shows the phylogenetic classification of DHPAAS sequences from insects.

FIG. 5-1 and FIG. 5-2 are diagrams for functional comparison of a wild-type DHPAAS and a variant DHPAAS, both from B. mori.

FIG. 6 is a kinetic analysis of H₂O₂ production from L-DOPA by the wild-type and the variant DHPAAS of B. mori.

FIG. 7 is a diagram showing in vitro production of dopamine, DHPAA and THP by the wild-type and the variant DHPAAS of B. mori.

FIG. 8 is a diagram illustrating the mechanism of THP production from L-DOPA by the variant DHPAAS.

FIG. 9-1 is a diagram showing in vivo production of dopamine, DHPAA and THP by DHPAAS. FIG. 9-2 is a diagram showing the result of chiral LC-MS analysis of the produced (R,S)-THP.

FIG. 10 is a diagram showing in vivo production of THP and reticuline.

FIG. 11 is a diagram showing in vivo production of THP, reticuline, and two intermediates.

FIG. 12 is a diagram showing in vivo production of THP and dopamine.

FIG. 13 is a diagram showing in vivo production of norcoclaurine.

FIG. 14 is a diagram showing in vivo production of norcoclaurine.

FIG. 15 is a diagram showing in vivo production of 4-HPAA, L-DOPA (Dopa), THP, norcoclaurine, and reticuline.

FIG. 16 is a diagram showing the amounts of 4-HPAA, L-DOPA, THP, norcoclaurine, and reticuline in vivo produced in the schemes of FIG. 15.

FIG. 17 is a diagram showing in vivo production schemes of THP, 3HNMC, and reticuline.

FIG. 18 is a diagram showing the amounts of in vivo THP, 3HNMC, and reticuline produced in the schemes of FIG. 17.

### DESCRIPTION OF EMBODIMENTS

Hereinafter, a recombinant host cell for producing benzylisoquinoline alkaloid (BIA) and a novel method for producing benzylisoquinoline alkaloid (BIA) according to the present invention are described in detail. In this specification, unless otherwise specified, molecular biological methods such as the preparation of DNA or vectors can be performed according to the methods described in general experimental manuals known to those skilled in the art or methods equivalent thereto. The terms used herein, unless otherwise specified, are interpreted in the sense commonly used in the art. The benzylisoquinoline alkaloid (BIA) according to the present invention refers to a compound having a benzylisoquinoline structure. Examples of the benzylisoquinoline alkaloid (BIA) include, but are not limited to, tetrahydropapaveroline (THP), norcoclaurine, 3-hydroxycoclaurine, 3-hydroxy-N-methylcoclaurine, and reticuline in various plants.

<Recombinant Host Cell>

The recombinant host cell of the present invention is a recombinant host cell for producing benzylisoquinoline alkaloid (BIA), in particular tetrahydropapaveroline (THP), norcoclaurine, 3-hydroxycoclaurine, 3-hydroxy-N-methylcoclaurine and/or reticuline, expressing a wild-type or a variant of aromatic aldehyde synthase (AAS), or a wild-type or a variant of aromatic amino acid decarboxylase (AAAD). Hereinafter, the recombinant host cell of the present invention will be described in detail.

The aromatic aldehyde synthase (AAS) expressed by the recombinant host cell of the present invention refers to a bifunctional enzyme that catalyzes decarboxylation and amino group oxidation of an aromatic amino acid. Specifically, the enzyme has a function of catalyzing the conversion from L-DOPA or tyrosine to dopamine and DHPAA or 4-HPAA. The obtained dopamine and DHPAA or 4-HPAA react with each other to generate THP or norcoclaurine. According to phylogenetic analysis, AAS is believed to be an enzyme that has diverged from aromatic amino acid decarboxylase (AAAD, EC 4.1.1.28). Both enzymes have structural similarities and are common in that they rely on pyridoxal 5′-phosphate (PLP) as a cofactor.

The AAS according to the present invention is not particularly limited as long as it has the above-described functions. Examples thereof include plant-derived AAS studied in plants and classified by KEGG such as phenylacetaldehyde synthase (PAAS, KEGG EC 4.1.1.109) and 4-hydroxyphenylacetaldehyde synthase (4-HPAAS, KEGG EC 4.1.1.108); 3,4-dihydroxyphenylacetaldehyde synthase (DHPAAS, KEGG EC 4.1.1.107) enzymes derived from insects; and others, such as indole-3-acetaldehyde synthase (IAAS). It should be noted that the species is not limited thereto, and includes various species including animals, plants, and bacteria. 3,4-dihydroxyphenylacetaldehyde synthase catalyzes decarboxylation and amino group oxidation of L-DOPA to produce DHPAA. Under some conditions, it also may catalyze decarboxylation of L-DOPA to produce dopamine to some extent. As AAS, from the viewpoint of the efficiency of THP conversion from L-DOPA, DHPAAS derived from insects is preferred among the above. It is believed that DHPAAS derived from insects has higher binding specificity to L-DOPA, resulting in higher efficiency in DHPAA production and dopamine (DA) production. As AAS, 4-HPAAS derived from plants is also preferred from the viewpoint of the efficiency of norcoclaurine conversion from tyrosine.

Examples of insect DHPAAS include that of Bombyx mori, Camponotus floridanus, Apis mellifera, Aedes aegypti, and Drosophila melanogaster. Among these, Bombyx mori DHPAAS is preferred from the viewpoint of the effect of the present invention.

Examples of plant AAS include that of Papaver somniferum, Arabidopsis thaliana, Arabidopsis lyrata, Brassica rapa, Camelina sativa, Corchorus olitorius, Brassica oleracea, Brassica cretica, Brassica napus, Capsella rubella, Eutrema salsugineum, Parasponia andersonii, Petroselinum crispum A, Prunus avium, Prunus yedoensis, Prunus dulcis, Prunus mume, Prunus persica, Prunus yedoens, Raphanus sativus, Tarenaya hassleriana, Trema orientale, Ziziphus jujuba, Malus domestica, Eriobotrya japonica, Corchorus capsularis, Morus notabilis, Pyrus x bretschneideri, Populus alba, Juglans regia, Citrus unshiu, Citrus sinensis, Quercus suber, Cephalotus follicularis, Eucalyptus grandis, Fragaria vesca, Populus trichocarpa, Durio zibethinus, Manihot esculenta, Durio zibethinus, Populus trichocarpa, Juglans regia, Manihot esculenta, Hevea brasiliensis, Citrus sinensis, Eucalyptus grandis, Durio zibethinus, Manihot esculenta, Hevea brasiliensis, Citrus clementina, Morus notabilis, Carica papaya, Rosa chinensis, Vitis vinifera, Populus euphratica, Rosa chinensis, Vitis vinifera, Actinidia chinensis, Populus euphratica, Ipomoea nil, and Petunia hybrida. Among these, Papaver somniferum AAS is preferred from the viewpoint of the effect of the present invention.

Examples of the microorganism AAAD and AAS include that of Pseudomonas putida (P. putida) and Methanocaldococcus jannaschii. Among these, Pseudomonas putida (P. putida) is preferred from the viewpoint of the effect of the present invention.

The AAS according to the present invention is preferably a variant of AAS in which an amino acid residue in the vicinity of the active center is substituted with the amino acid residue found in DOPA decarboxylase (DDC).

Specifically, for example, with respect to DHPAAS derived from insects, mutations of Phe79Tyr, Tyr80Phe, Asn192His are preferred, and the variant may have any one of these mutations, may have any two of them, or may have all of the three mutations. Among these, from the viewpoint of the efficiency of THP conversion from L-DOPA, Phe79Tyr-Tyr80Phe-Asn192His DHPAAS having all of the three mutations, Phe79Tyr-Tyr80Phe DHPAAS having two mutations of Phe79Tyr-Tyr80Phe, Asn192His DHPAAS having only a mutation of Asn192His are preferred, and Phe79Tyr-Tyr80Phe-Asn192His DHPAAS and Asn192His DHPAAS are more preferred.

The aromatic amino acid decarboxylase (AAAD) expressed by the recombinant host cell of the present invention refers to an enzyme that catalyzes decarboxylation of an aromatic amino acid. Specifically, the enzyme is an enzyme having a function of catalyzing the conversion from L-DOPA or tyrosine to dopamine or 4-HPAA. Specific examples thereof include tyrosine decarboxylase (TyDC), DOPA decarboxylase (DDC), phenylalanine decarboxylase (PDC), and tryptophan decarboxylase (TDC).

Preferred examples of the species of the aromatic amino acid decarboxylase (AAAD) expressed by the recombinant host cell of the present invention can include a species similar to the species described for the AAS described above.

When the aromatic amino acid decarboxylase (AAAD) expressed by the recombinant host cell of the present invention is TyDC1 derived from a plant, mutations of Phe99Tyr, Tyr98Phe, Leu205Asn are preferred, and the variant may have any one of these mutations, may have any two of them, or may have all of the three mutations. Among these, from the viewpoint of the efficiency of norcoclaurine conversion from tyrosine, Phe99Tyr-Tyr98Phe-Leu205Asn TyDC1 having all of the three mutations is preferred. Meanwhile, when the AAAD is TyDC3, mutations of Phe101Tyr, Tyr100Phe, His203Asn are preferred, and the variant may have any one of these mutations, may have any two of them, or may have all of the three mutations. Among these, from the viewpoint of the efficiency of norcoclaurine conversion from tyrosine, Phe101Tyr-Tyr100Phe-His203Asn TyDC3 having all of the three mutations is preferred.

The 79th, 80th, and 192th active site residues of DHPAAS of the insect Bombyx mori are structurally conserved throughout in aromatic amino acid decarboxylase (AAAD), aromatic aldehyde synthase (AAS), DHPAAS, and other related proteins. However, the numbering of residues varies by species depending on the size of the protein. For example, Phe79 of DHPAAS of Bombyx mori corresponds to Tyr79 of DDC of Pseudomonas putida, Tyr98 of TyDC1 of Papaver somniferum, and Tyr100 of TyDC3 of Papaver somniferum. Tyr80 of DHPAAS of Bombyx mori corresponds to Phe80 of DDC of Pseudomonas putida, Phe99 of TyDC1 of Papaver somniferum, and Phe101 of TyDC3 of Papaver somniferum. Asn192 of DHPAAS of Bombyx mori corresponds to His181 of DDC of Pseudomonas putida, Leu205 of TyDC1 of Papaver somniferum, and His203 of TyDC3 of Papaver somniferum. It should be noted that, for example, Papaver somniferum has other TyDCs including TyDC2 and 4 to 9, and here Leu205 of TyDC1 corresponds to His205 of TyDC5, TyDC6, TyDC8, and TyDC9, and His203 of TyDC2 and TyDC7.

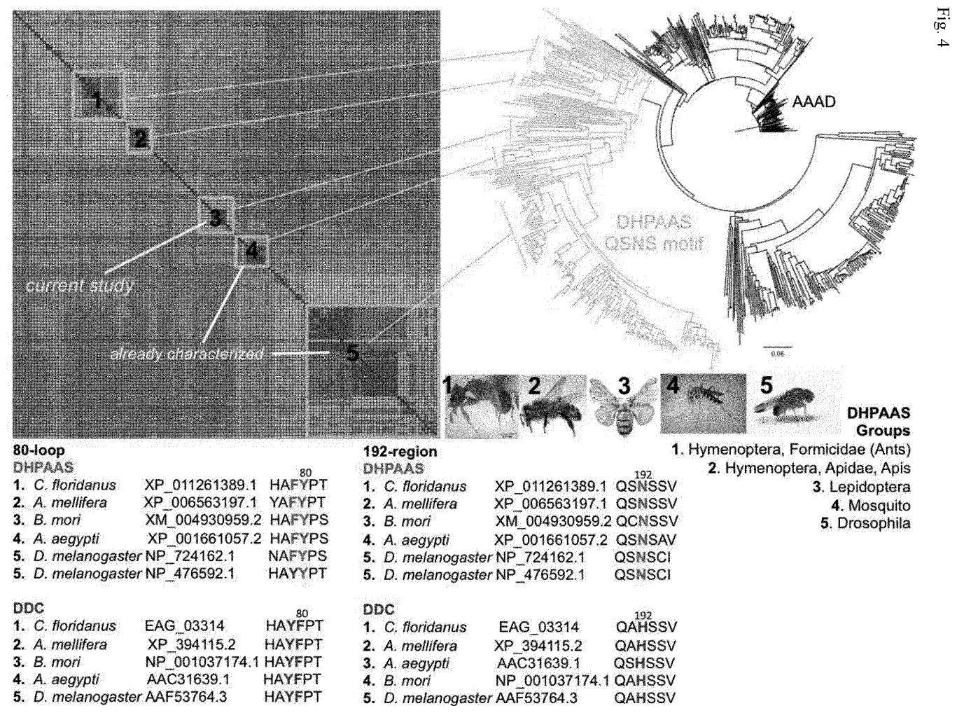

When the numbering of amino acid residues of 3,4-dihydroxyphenylacetaldehyde synthase (DHPAAS) from Bombyx mori is referred to herein, the present invention applies to all amino acid positions corresponding to the structurally conserved residues described above. To identify the structurally conserved residue, reference can be made to structural diagrams. Reference can also be made to sequence alignment diagrams for amino acid numbering different cases at corresponding positions (FIGS. 3 and 4).

The recombinant host cell of the present invention contains a gene encoding the AAS (wild-type and various variants) described above. Examples of such gene include genes having nucleotide sequences represented by SEQ ID NO: 1 (DHPAAS wild-type), SEQ ID NO: 2 (Asn192His DHPAAS variant), SEQ ID NO: 3 (Phe79Tyr-Tyr80Phe DHPAAS variant), SEQ ID NO: 4 (Phe79Tyr-Tyr80Phe-Asn192His DHPAAS variant) for DHPAAS derived from insects. The amino acid sequences of the corresponding proteins are represented by SEQ ID NO: 5 (DHPAAS wild-type), SEQ ID NO: 6 (Asn192His DHPAAS variant), SEQ ID NO: 7 (Phe79Tyr-Tyr80Phe DHPAAS variant), SEQ ID NO: 8 (Phe79Tyr-Tyr80Phe-Asn192His DHPAAS variant), respectively. In order to improve the efficiency of protein production of the wild-type and variant DHPAAS in the recombinant host cell of the present invention, a SUMO tag expression system can be used. As the amino acid sequence used in the system, a sequence represented by SEQ ID NO: 9 (DHPAAS wild-type), SEQ ID NO: 10 (Asn192His DHPAAS variant), SEQ ID NO: 11 (Phe79Tyr-Tyr80Phe DHPAAS variant), SEQ ID NO: 12 (Phe79Tyr-Tyr80Phe-Asn192His DHPAAS variant) can be employed, respectively.

Thus, the AAS gene carried by the recombinant host cell of the present invention, in the case of DHPAAS, is preferably any DNA of (a), (b) or (c) below.

(a) a DNA consisting of a nucleotide sequence of any one of SEQ ID NOs: 1 to 4.
(b) a DNA that hybridizes with a DNA consisting of a nucleotide sequence complementary to the DNA consisting of the nucleotide sequence of (a) under a stringent condition, and encodes a protein having an enzymatic activity (difunctional) of DHPAAS.
(c) a DNA consisting of a nucleotide sequence that has 70% or more, preferably 80% or more, more preferably 90% or more, further preferably 95% or more, particularly preferably 98% or more homology to any one of the nucleotide of SEQ ID NOs: 1 to 4, having the mutation described above introduced into the wild-type sequence, and encoding a protein which has the enzymatic activity (difunctional) of DHPAAS.

The recombinant host cell of the present invention contains a gene encoding the aromatic amino acid decarboxylase (AAAD) described above. As examples of such a gene, when the aromatic amino acid decarboxylase (AAAD) is TyDC1 derived from a plant, the wild-type includes one having the amino acid sequence of SEQ ID NO: 15 and the nucleotide sequence of SEQ ID NO: 16 corresponding thereto. By using the primers of SEQ ID NO: 17 and SEQ ID NO: 18, a nucleotide into which the mutations Phe99Tyr, Tyr98Phe have been introduced can be synthesized. Furthermore, by using the primers of SEQ ID NO: 19 and SEQ ID NO: 20, a nucleotide into which the mutation Leu205Asn has been introduced can be synthesized. When the aromatic amino acid decarboxylase (AAAD) of the recombinant host cell of the present invention is TyDC3 derived from a plant, the wild-type includes one having the amino acid sequence of SEQ ID NO: 21 and the nucleotide sequence of SEQ ID NO: 22 corresponding thereto. By using the primers of SEQ ID NO: 23 and SEQ ID NO: 24, a nucleotide into which the mutations of Phe101Tyr, Tyr100Phe have been introduced can be synthesized. Furthermore, by using the primers of SEQ ID NO: 25 and SEQ ID NO: 26, a nucleotide into which the mutation His203Asn has been introduced can be synthesized.

It is preferable that the recombinant host cell of the present invention contain, in addition to a gene encoding the AAS (wild-type and various variants) or AAAD (wild-type and various variants) described above, a gene encoding an enzyme necessary for synthesizing reticuline from THP or norcoclaurine.

Examples of such enzymes include norcoclaurine synthase (NCS). Norcoclaurine synthase (NCS) is an enzyme that synthesizes norcoclaurine and THP from dopamine and DHPAA, or dopamine and 4-HPAA. It is preferable that the recombinant host cell of the present invention contain a gene encoding norcoclaurine synthase (NCS).

Further examples of such enzymes include norcoclaurine 6-O-methyltransferase (6′OMT), 3′-hydroxy-N-methyl-(S)-coclaurine-4′-O-methyltransferase (4′OMT), coclaurine-N-methyltransferase (CNMT), and N-methylcoclaurine 3-hydroxylase (NMCH). It is more preferred that the recombinant host cell of the present invention have all the genes encoding norcoclaurine 6-O-methyltransferase (6′OMT), 3′-hydroxy-N-methyl-(S)-coclaurine-4′-O-methyltransferase (4′OMT), and coclaurine-N-methyltransferase (CNMT).

The stringent conditions refer to conditions in which only specific hybridization occurs and non-specific hybridization does not occur. Such conditions are typically conditions of about 6 M urea, 0.4% SDS, and 0.5×SSC. The DNA obtained by hybridization preferably has a high homology of 60% or more to the DNA consisting of the nucleotide sequence of (a) above, and further preferably has a homology of 80% or more.

The homology means a degree of similarity between sequences of two polypeptides or polynucleotides, and is determined by comparing two sequences aligned to an optimal state (a state in which sequence match is maximum) across the region of the amino acid sequence or nucleotide sequence to be compared. The numerical value (%) of homology is calculated by determining the same amino acid or nucleotide present in both (amino acid or nucleotide) sequences to determine the number of matching sites, then dividing the number of matching sites by the total number of amino acids or nucleotides in the sequence region to be compared, and multiplying the obtained numerical value by 100. Examples of the algorithm for obtaining optimal alignment and homology include various algorithms typically available to those skilled in the art (e.g., a BLAST algorithm, a FASTA algorithm). The homology of amino acid sequences is determined, for example, using sequence analysis software such as BLASTP or FASTA. The homology of nucleotide sequences is determined using software such as BLASTN or FASTA.

The genes can be obtained by PCR or hybridization techniques well known to those skilled in the art, or by artificial synthesis methods using a DNA synthesizer or the like. Gene sequence determination can be performed by methods well known to those skilled in the art with a sequencer.

The host cell used in the present invention may be any host cell well known to those skilled in the art, including a prokaryotic cell and a eukaryotic cell, such as a bacterial cell, a fungal cell, a yeast cell, a mammalian cell, an insect cell, or a plant cell. Examples of the bacterial cell include cells of any species of Escherichia, Salmonella, Streptomyces, Pseudomonas, Staphylococcus, or Bacillus, including, for example, Escherichia coli (E. coli), Lactococcus lactis, Bacillus subtilis, Bacillus cereus, Salmonella typhimurium, and Pseudomonas fluorescens.

As the host cell used in the present invention, E. coli cells are preferred because E. coli cells are resistant to various stresses and easily genetically transformed.

In the present invention, the term “polynucleotide” means both a single nucleic acid and a plurality of nucleic acids, and includes nucleic acid molecules such as mRNA, plasmid RNA, full-length cDNA, and fragments thereof. The polynucleotide is composed of any polyribonucleotide or polydeoxyribonucleotide and may be modified or unmodified. The polynucleotide may be a single-stranded or double-stranded or may be a mixture of both.

In the present invention, the term “heterologous” in the “recombinant host cell for producing a benzylisoquinoline alkaloid (BIA), expressing a wild-type or a variant of an aromatic aldehyde synthase (AAS) of a heterologous species” refers to a cell expressing a protein derived from a species different from that of the recombinant host cell of the present invention, or a polynucleotide encoding the protein. For example, when the recombinant host cell of the present invention is an E. coli cell, the heterologous protein and the heterologous polynucleotide include a protein and a polynucleotide derived from an insect, a plant, or the like. An object of introducing a polynucleotide encoding a heterologous protein in the recombinant host cell of the present invention is to introduce a polynucleotide encoding a protein such as an enzyme not originally possessed by the host cell from the heterologous, and to function a metabolic pathway of interest, i.e., a metabolic pathway that produces THP and/or reticuline from L-DOPA.

(Method of Introducing Polynucleotide)

In order to express a wild-type or a variant of aromatic aldehyde synthase (AAS) of a heterologous species in a host cell, it is necessary to express a polynucleotide encoding the wild-type or the variant of aromatic aldehyde synthase (AAS) of a heterologous species in the host cell. The cell may be transformed, for example, with an expression vector containing the polynucleotide. The same applied to the case for expressing a polynucleotide encoding an enzyme required to synthesize reticuline from THP. The expression vector is not particularly limited as long as the expression vector contains the gene of the present invention in an expressible state, and a vector suitable for each host can be used.

The expression vector of the present invention can be prepared by inserting a transcriptional promoter upstream, and possibly a terminator downstream of the heterologous polynucleotide described above to construct an expression cassette, and inserting the cassette into an expression vector. Alternatively, when a transcription promoter and/or terminator is already present in an expression vector, the expression vector of the present invention can be prepared, without constructing an expression cassette, by utilizing the promoter and/or terminator in the vector and inserting the heterologous polynucleotide therebetween.

To insert the heterologous polynucleotide into the vector, a method using a restriction enzyme, a method using a topoisomerase, or the like can be used. An appropriate linker may be added when it is necessary for insertion. Ribosomal binding sequences such as a SD sequence and a Kozak sequence are known as nucleotide sequences important for translation into amino acids, and these sequences may be inserted upstream of the gene. Along with insertion, a portion of the amino acid sequence encoded by the gene may be replaced.

The vector used in the present invention is not particularly limited as long as the vector carries the gene of the present invention, and a vector suitable for each host can be used. Examples of the vector include a plasmid DNA, a bacteriophage DNA, a retrotransposon DNA, and an artificial chromosomal DNA.

The method for introducing the expression vector into a host is not particularly limited as long as the method is suitable for the host. Examples of the available methods include an electroporation method, a method using a calcium ion, a spheroplast method, a lithium acetate method, a calcium phosphate method, and a lipofection method. The expression of the polynucleotide in a recombinant host cell can be quantified according to methods known to those skilled in the art. For example, the quantity of the expression can be expressed by the percent of the polypeptide encoded by the polynucleotide relative to the total cell proteins. The quantity can also be confirmed using a cell extract of the transformed cell by western blotting with an antibody capable of detecting the polypeptide encoded by the polynucleotide, or by real-time PCR or the like with a primer that specifically detects the polynucleotide.

<Production Method of Benzylisoquinoline Alkaloid (BIA) of the Present Invention>

The present invention also provides a method for producing tetrahydropapaveroline (THP), norcoclaurine, 3-hydroxycoclaurine, 3-hydroxy-N-methylcoclaurine and/or reticuline using the recombinant host cell of the present invention described above. As the method for producing the present invention, there are two main methods.

One method is a method including a step of culturing the recombinant host cell of the present invention in a L-DOPA and/or tyrosine-containing culture medium. The recombinant host cell of the present invention into which L-DOPA and/or tyrosine in the medium are incorporated can efficiently produce THP, norcoclaurine, 3-hydroxycoclaurine, 3-hydroxy-N-methylcoclaurine and/or reticuline using AAS or the like expressed in the cell. The produced THP, norcoclaurine, 3-hydroxycoclaurine, 3-hydroxy-N-methylcoclaurine and/or reticuline are secreted into the culture medium.

Another method is a method including a step of causing a wild-type or a variant of an aromatic aldehyde synthase (AAS) or an aromatic amino acid decarboxylase (AAAD) to act on L-DOPA and/or tyrosine in a cell-free system. In this method, for example, the wild-type or the variant of an aromatic aldehyde synthase (AAS) or an aromatic amino acid decarboxylase (AAAD) acts directly on L-DOPA and/or tyrosine to produce dopamine, and phenylaldehyde such as DHPAA or 4-HPAA in vitro, and dopamine and DHPAA or 4-HPAA bond to each other to generate THP or norcoclaurine. Furthermore, reticuline is produced by causing an enzyme necessary for the synthesis of reticuline from THP or norcoclaurine to act on THP or norcoclaurine. Here, it is preferable that an enzyme obtained from the recombinant host cell of the present invention described above be used as the wild-type or the variant of an aromatic aldehyde synthase (AAS) or an aromatic amino acid decarboxylase (AAAD).

### Examples

Hereinafter, the present invention will be described in detail with Examples, but the present invention is not limited by the Examples. In some drawings for describing the Examples, some amino acids are represented by one letter notation.

1. Selection of Symmetric THP Production Pathway Via DHPAAS for Reticuline Biosynthesis

M-path enzyme search was performed according to the method of Araki et al. (Araki, et al. M-path: a compass for navigating potential metabolic pathways. Bioinformatics 31, 905-911 (2015)) using a web-based version. The M-path score was calculated as a Tanimoto coefficient. For the M-path database, the 2016 version updated to the latest substrate, product, and enzyme information from KEGG was used. The curation mode was employed to explore enzymes that mediate from tyrosine (PubChem CID: 6057) to 4-HPAA (CID: 440113), from L-DOPA (CID: 6047) to DHPAA (CID: 119219), from tyrosine to 2′-norberbamunine (CID: 441063), from histidine (CID: 6274) to imidazole-4-acetaldehyde (CID: 150841), and from 4-aminophenylalanine (CID: 151001) to 4-aminophenylacetaldehyde (CID: 20440853). The original mode was employed in M-path for conversion from tyrosine to homovanillic acid (CID: 1738).

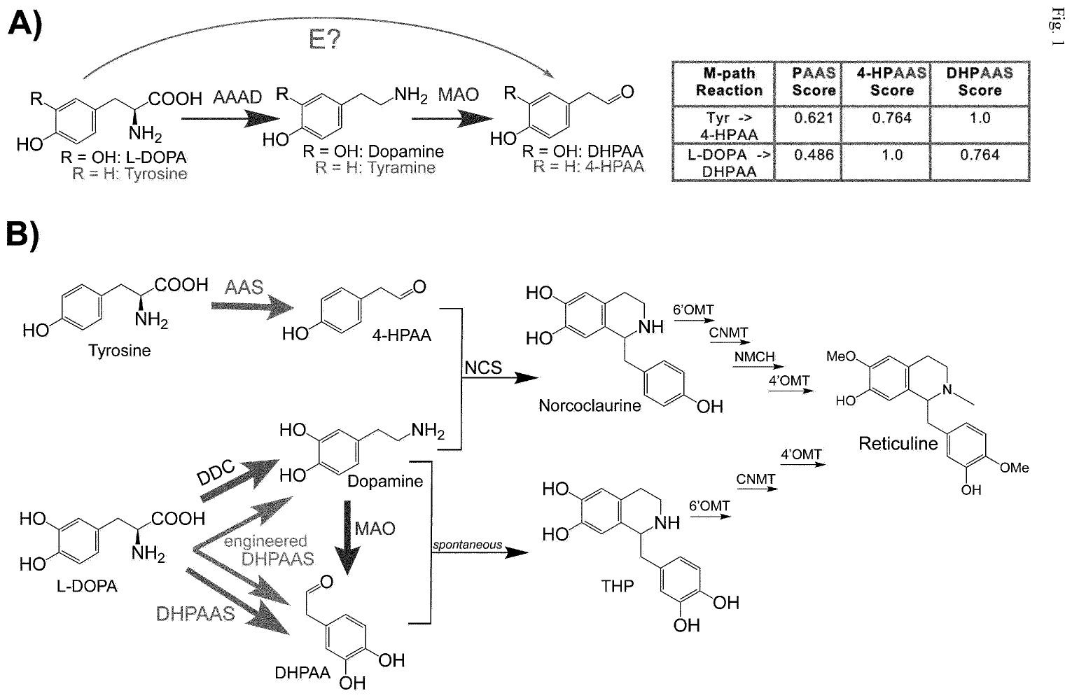

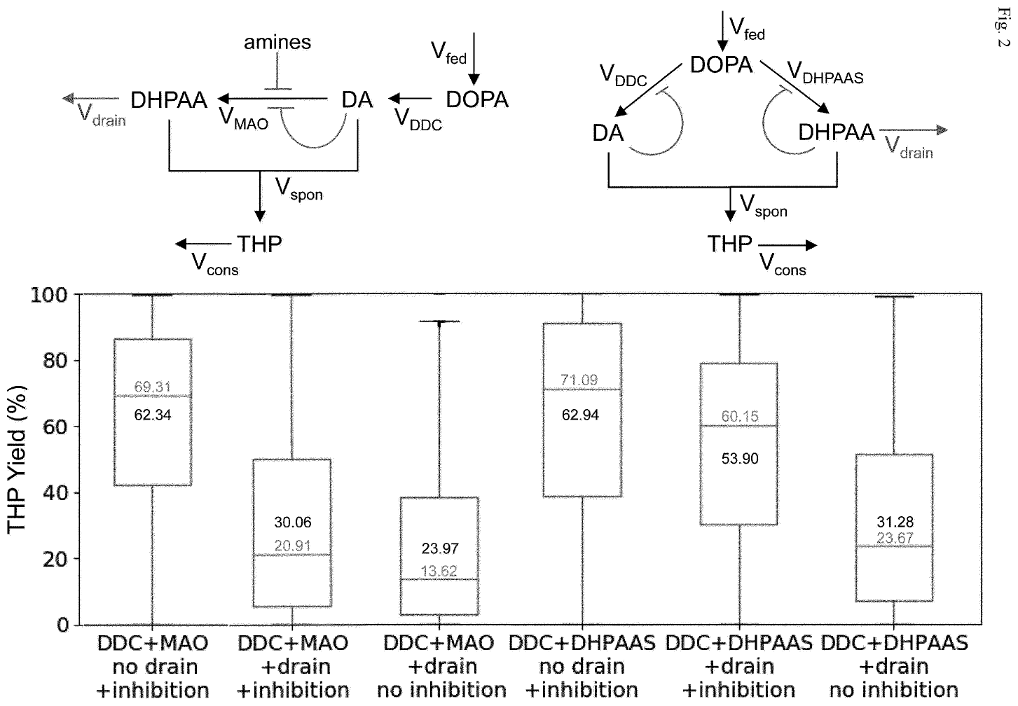

M-path enzyme search is advantageous in that, unlike exploration of known enzyme networks, unknown enzyme reactions can be predicted based on the substrate and product similarities. To explore optimization of BIA production from L-DOPA, an M-path enzyme search algorithm was tested according to the method of Araki et al. When M-path was used in a database combining the latest enzymes databases from BRENDA (https://www.brenda-enzymes.org/) and Kyoto Encyclopedia Genes and Genomes (KEGG, http://www.kegg.jp), an insect-derived 3,4-dihydroxyphenylacetaldehyde synthases (DHPAAS) and a plant-derived aromatic aldehyde synthases (AAS; PAAS, 4-HPAAS) were identified as presumed shortcuts for the production of 4-hydroxyphenyl acetaldehyde (4-HPAA or 4-HPA) from L-tyrosine (Tyr) and the production of 3,4-dihydroxyphenyl acetaldehyde (DHPAA, DHPA or DOPAL) from 3,4-dihydroxyphenylalanine (L-DOPA) (FIG. 1A). Furthermore, by combining the DHPAAS or aromatic aldehyde synthase (AAS; PAAS, 4-HPAAS) with 3,4-dihydroxyphenyl alanine decarboxylase (DDC), novel and symmetric THP and norcoclaurine production pathways different from the conventionally reported MAO-mediated pathway were found (FIG. 1B).

The aromatic aldehyde synthase (AAS; PAAS, 4-HPAAS) and DHPAAS are bifunctional enzymes that catalyze decarboxylation and amino group oxidation of aromatic amino acids. These enzymes, including phenylacetaldehyde synthases (PAAS, KEGG EC 4.1.1.109) and 4-hydroxyphenylacetaldehyde synthases (4-HPAAS, KEGG EC 4.1.1.108), found in plants, are collectively referred to as AAS. The enzyme DHPAAS (EC 4.1.1.107), recently found from insects, catalyzes oxidative decarboxylation of L-DOPA, and is thus considered as an AAS-related protein. The “AAS”, in the broad sense, is an aromatic aldehyde synthase and includes both an insect-derived 3,4-dihydroxyphenylacetaldehyde synthase (DHPAAS) and a plant-derived aromatic aldehyde synthase (AAS; PAAS, 4-HPAAS), but, in the narrow sense, refers to an aromatic aldehyde synthase derived from a plant due to the history of enzyme discovery. The phylogenetic analysis shows that AAS derived from plants and DHPAAS derived from insects described above are diverged from aromatic amino acid decarboxylase (AAAD, EC 4.1.1.28). Thus, the AAS, DHPAAS and AAAD described above have structural similarities and rely on pyridoxal 5′-phosphate (PLP) as a cofactor. The AAS and DHPAAS described above have been assigned to EC 4.1.1.—by KEGG, but it is not easy to classify them due to their bifunctional action, and some unclarity remains in these relatively newly-characterized enzymes.

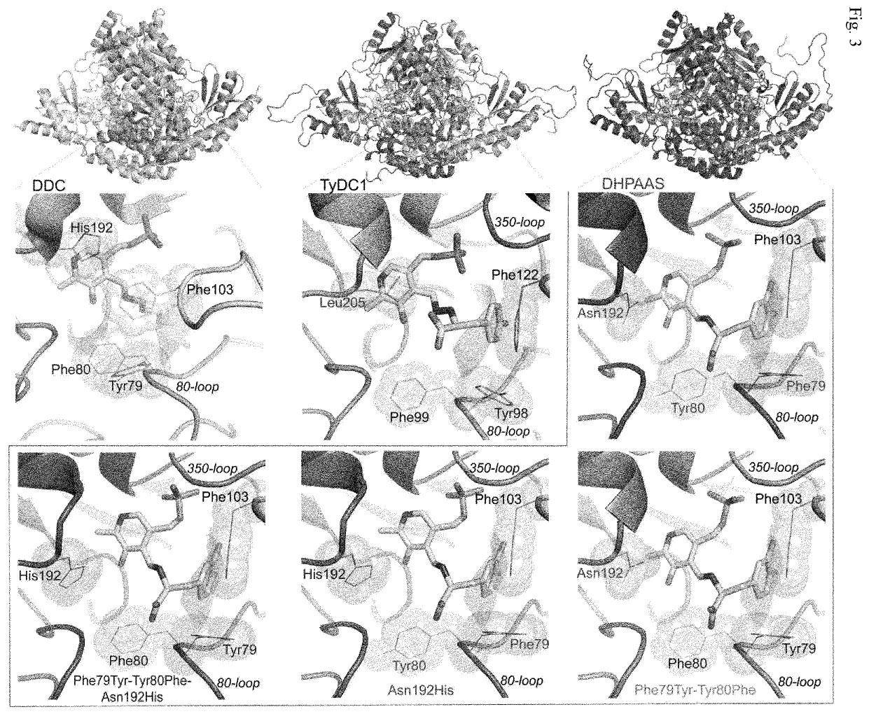

The AAS and DHPAAS-mediated symmetric BIA production pathways have advantages over the MAO-mediated asymmetric pathway (FIG. 1). Such advantages include that the soluble DHPAAS has higher specificity to L-DOPA than MAO. Mathematical models and numerical simulations were used to compare THP productions by the asymmetric (DDC-MAO) pathway and the symmetric (DDC-DHPAAS) pathway, as shown in FIG. 2. In the asymmetric pathway, competitive inhibition from other substrates was introduced into the MAO reaction rate V_{MAO} because MAO recognizes various amines. In the symmetric pathway, two models were constructed: one without feedback and one with feedback inhibition in the calculation of the reaction rates of DDC (VDDC) and DHPAAS (VDHPAAS). The range of possible parameter values in the models was obtained with reference to Placzek, S. et al. BRENDA in 2017: new perspectives and new tools in BRENDA. Nucleic Acids Res. 45, D380-D388 (2017). To predict the performance of each pathway, parameter values were randomly generated within the relevant ranges, and Monte Carlo simulations were performed. The number of iterations was 10,000 times, and the simulation time was 0 to 50 hours. L-DOPA was supplied as a constant term based on the randomly generated parameters. When the maximum amount of 100 mM L-DOPA was reached, the supply of the substrate to the system was stopped. The handmade program was run on Python 3.0 using scipy.integrate.odeint as the solver for the numerical simulation.

From the results of in vitro and in vivo tests below, it was suggested that highly reactive DHPAA was degraded and depleted by the reaction with competing nucleophilic reagents present in the cells or growth medium. Inclusion of the disappearance of DHPAA in the dynamic model resulted in slightly lower THP yields (FIG. 2), which was in good agreement with experimental yields. However, many diverse variables including buffer composition, pH, temperature, potential inhibitors, and metabolic flux of the growth medium should also be considered as training data for improving THP yields. When both of the feedback inhibition by the product and the disappearance of DHPAA were incorporated into the calculations, the symmetric DDC-DHPAAS pathway showed much higher predicted yields of THP than MAO-mediated asymmetric pathway (FIG. 2). These models suggest that DHPAAS-mediated pathway may produce THP at higher levels than the previously reported amount of MAO-mediated THP produced (maximum 1 mM). Furthermore, from the feedback inhibition model, it can be found that the balance between dopamine and DHPAA is important for optimal THP production. Thus, the balance regulation between DHPAA production and dopamine production by DHPAAS was further investigated.

2. Structure-Based Identification and Engineering of Novel DHAAS Variants

To select the optimal sequence for BIA production, putative structures of plant-derived AAS and insect-derived DHPAAS were compared. Dimeric homology models of putative AAS or DHPAAS complexed with an aromatic amino acid substrate covalently attached to a PLP cofactor were created with MODELLER operating in Chimera, and the structures were improved with MOE (FIG. 3).

M-Path identified 4-HPAAS as an enzyme that produces 4-HPAA, an important intermediate in plant BIA synthesis. Then, with assumption that Papaver somniferum (P. somniferum) utilizes AAS activity for native 4-HPAA biosynthesis, potential AAS enzymes were searched in the sequence of P. somniferum. Interestingly, Papaver somniferum (P. somniferum) tyrosine decarboxylase (TyDC1) modeled based on the structure of Sus Scrofa DDC complexed with carbidopa (PDB ID: 1JS3) contains a novel isoleucine residue at the position corresponding to the AAAD active site His192 (FIG. 3, middle panel), which is noted as an important catalyst residue. However, except for the novel TyDC1 Leu205, the entire TyDC1 sequence of P. somniferum is very similar to the standard AAAD sequence. In contrast, comparison with putative insect DHPAAS sequences shows more apparent difference in active sites (FIG. 3). Thus, the focus was shifted to insect DHPAAS to select the optimal BIA production system.

Many questions remain to be answered concerning the evolution of insect DHPAAS and the oxidative decarboxylation mechanism of DHPAAS, including the elucidation of all essential catalyst residues. To clarify these questions and to gain insights based on the mechanism of DHPAAS, phylogenetic classification was performed in combination with structural analysis.

Dimeric homology models of DHPAAS of Bombyx mori (B. mori) and TyDC1 of Papaver somniferum (P. somniferum) were produced with MODELLER and Chimera. The crystal structures of DDC (PDB ID: 3K40) and histidine decarboxylase (4E10) of D. melanogaster were used as templates for modeling of DHPAAS of B. mori. The structure of DDC of Sus Scrofa complexed with carbidopa (PDB ID: 1JS3) was used as a template for TyDC1. Refinements of the covalent attachment of PLP to the aromatic amino acid substrate and the structure were performed using Molecular Operating Environment (MOE). The finished structure was analyzed by PyMOL.

The AAAD and AAS sequences of insects were collected from the protein BLAST non-duplicate database by searching from the insect sequences NP_476592.1, NP_724162.1, XP_319838.3, EDS39158.1, EAT37246.1 and EAT37247.1. Sequences duplicated and sequences having more than 700 amino acids were removed, and the resulting sequences were aligned to create a phylogenetic tree using split value 0.12. Clusters were identified by creating a sequence identity table using MOE. By phylogenetic analysis of 738 AAAD-associated sequences of insects, putative 247 DHPAAS sequences and 5 DHPAAS groups were identified (FIG. 4).

Property-unknown Lepidopteran DHPAAS constituting the central phylogenetic group (FIG. 4) was selected to obtain new findings for the DHPAAS mechanism. In analyzing the structure of insect DHPAAS, a novel loop formed by Gly353-Arg324 could not be easily modeled even using the structure of Drosophila melanogaster 3,4-dihydroxyphenylalanine decarboxylase (DDC, PDB ID: 3K40) as a template. This 320-350 loop involved in cross-dimer active site formation and substrate binding was better modeled using human histidine decarboxylase in a complex with histidine methyl ester (PDB ID: 4E1O) as a template.

Comparison of DDC and DHPAAS active sites revealed that position 192 (in the numbering of B. mori and D. melanogaster DHPAAS) is an important residue in determining catalytic activity of decarboxylase or aldehyde synthase (FIGS. 3 and 4). This 192 residue can make a hydrogen bond with an external aldimine of the PLP-aromatic amino acid complex oxidized by the AAS mechanism. Although the properties of Aedes aegypti and Drosophila melanogaster DHPAAS containing Asn192 have been previously reported, this study also identified and confirmed Asn192 in a separate way as an important catalytic site through structural and functional analysis.

With careful comparison of the structures of DDC and DHPAAS, it was shown that Phe79 and Tyr80 of DHPAAS play an additional role in distinguishing DHPAAS activity from DDC activity (FIGS. 3 and 4). Tyr79-Phe80 is conserved in insect DDC, but this 79-80 motif is generally reversed as Phe79-Tyr80 in insect DHPAAS, and these residues also surround the external aldimine of the PLP-substrate complex (FIG. 3). Thus, we assumed that these residues are involved in the catalytic mechanism of DHPAAS and are useful for classification of DHPAAS. Of the identified five DHPAAS groups, Apis (honeybee) and mosquito have conserved Phe79-Tyr80. In the DHPAAS sequences of Drosophila, Phe79-Tyr80 is conserved in a sequence referred to as Isoform X1, and Tyr79-Tyr80 is conserved in a sequence referred to as Isoform X2 (including NP476592.126). In the DHPAAS group of Lepidoptera and Formicidae, Phe79-Tyr80, Tyr79-Tyr80, and Tyr79-Phe80 are mixed (FIG. 4).

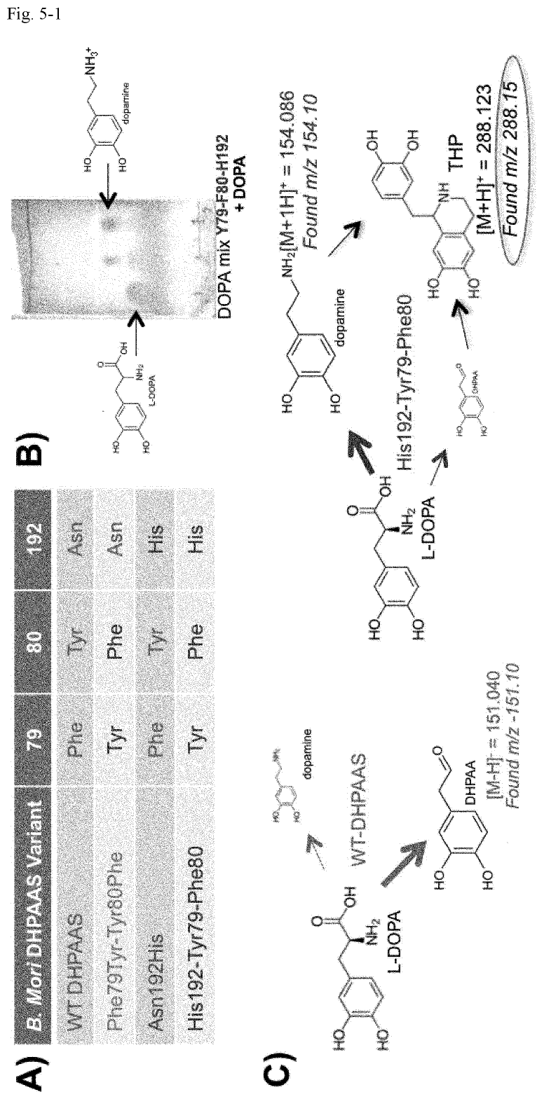

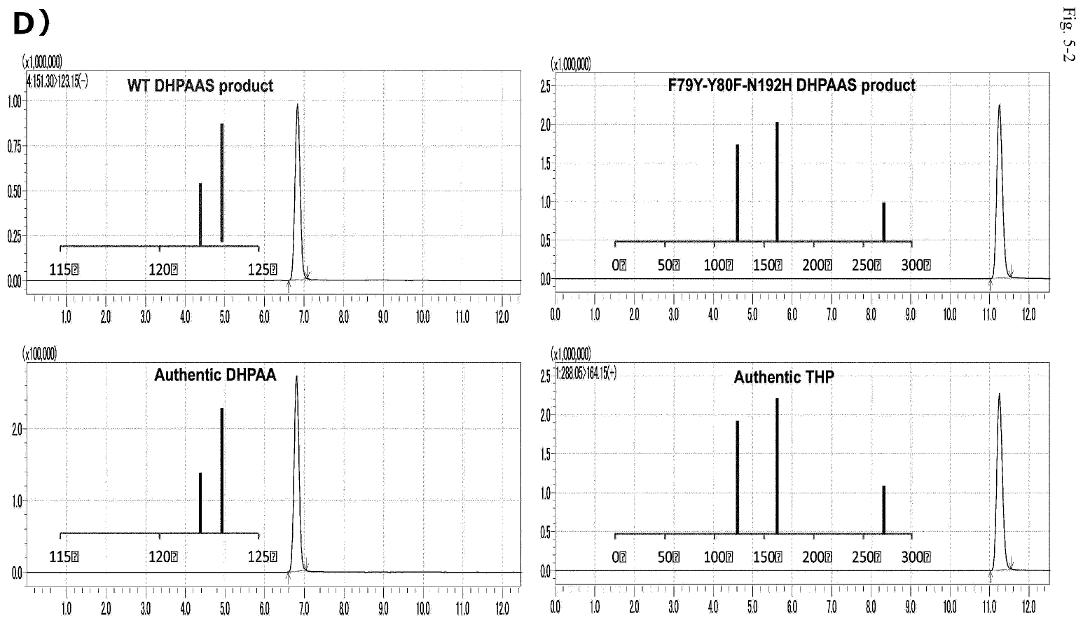

In the following experiments, B. mori sequence XM_004930959.2 was selected as a typical DHPAAS sequence that contains all three residues Phe79, Tyr80 and Asn192 specific for DHPAAS and also contains Gly353 reported for increased substrate specificity for L-DOPA. Additionally, amino acid variants of Phe79Tyr, Tyr80Phe, and Asn192His DHPAAS catalytic sites were designed to explore production regulatory mechanisms of dopamine and DHPAA (FIG. 5A).

3. Preparation of Recombinant B. mori DHPAAS

The cDNA sequence of full-length wild-type B. mori DHPAAS (XM_004930959.2; SEQ ID NO: 1) was synthesized with GeneArt (Invitrogen), and cloned into a pE-SUMO vector having kanamycin resistance (LifeSensors Inc.) via BsaI restriction enzyme sites. The cDNA of the amino acid variants (SEQ ID NOs: 2 to 4) were generated using overlap PCR. DHPAAS expression vectors were introduced into BL21 (DE3) maintained in LB medium supplemented with 50 μg/mL kanamycin, or BL21 (DE3) pLysS maintained in LB medium supplemented with 50 μg/mL kanamycin and 34 μg/mL chloramphenicol to transform. Expression of recombinant DHPAAS was induced by adding 0.2-0.45 mM IPTG to aerobically grown E. coli in LB medium. After induction, the culture temperature was reduced to 14-16° C. After incubation overnight, the cells were pelleted by centrifugation, resuspended in phosphate buffered saline (PBS), and lysed by ultrasound treatment while cooled on ice. The lysate was centrifuged, and the clarified lysate was applied to HiTrap TALON and HisTrap HP columns (GE Life Sciences), then washed with PBS and 10-20 mM imidazole. Recombinant DHPAAS was eluted with 450-1,000 mM imidazole. The buffer was then exchanged to PBS supplemented with PLP using a Millipore Amicon Ultra-15 centrifugal filter.

4. Analysis of DHPAAS Substrates and Reaction Products

Changes in L-DOPA and DHPAAS reactions were non-quantitatively analyzed for substrates and products by thin-layer chromatography (TLC). The TLC was performed on an aluminum plate coated with silica gel 60F254 (Merck Millipore). A mixture of 1-butanol:acetic acid:H₂O=7:2:1 ratio was used as the mobile phase. The components of the DHPAAS reaction were analyzed under UV, followed by heating to perform ninhydrin staining.

Substrates and products of the DHPAAS reaction were identified by mass spectra obtained with Shimadzu LCMS-8050 ESI triple quadrupoles. Quantitative analysis was performed using Shimadzu LCMS-8050 manipulated in Multiple Reaction Monitoring (MRM) mode with Nexera X2 UHPLC system. For L-DOPA (TCI), dopamine (TCI), DHPAA (Santa Cruz Biotechnology), and THP (Sigma), qualifier MRM transitions of 198.10>152.10 (+), 154.10>91.05 (+), 151.30>123.15 (−), and 288.05>164.15 (+) were used, respectively. For dopamine, DHPAA and THP, qualifier MRM transitions of 154.10>137.05 (+), 151.30>122.10 (−) and 288.05>123.15 (+) were used, respectively. For reticuline, a qualifier MRM transition of 330.10>177.20 (+) was used. Separation was performed with Discovery HS F5-3 column (3 μm, 2.1 mm×150 mm, Sigma-Aldrich) heated to 40° C., with a concentration gradient of 0.1% formic acid aqueous solution and 0.1% acetonitrile formic acid as the mobile phase, at 0.25 mL/min. Chiral analysis of (R,S)-THP was performed using the same LC-MS system, with heated Astec CYCLOBOND I 2000 column (5 μm, 2.1 mm×150 mm, Sigma-Aldrich), with a mobile phase gradient of 90% acetonitrile-50 mM NH₄OAc, pH 4.5, by eluting at eluting rate of 0.3 mL/min.

5. Functional Conversion of B. mori DHPAAS by Amino Acid Substitution

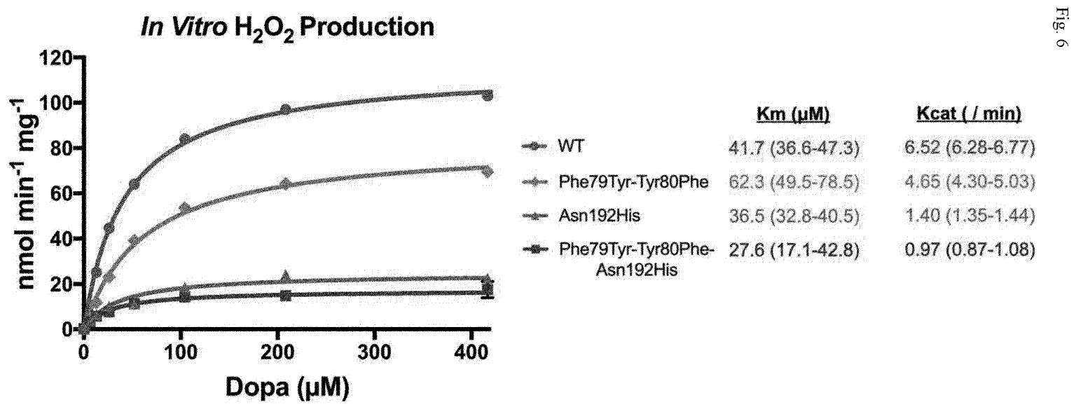

Recombinant B. mori XM_004930959.2 wild-type protein produced DHPAA as a main product with L-DOPA, as shown by detection of anion m/z 151.10 and lack of major dopamine ions (FIG. 5). This identification of B. mori DHPAAS suggests that the above analysis for the DHPAAS phylogenetic group is accurate. From the results of structural analysis, a hypothesis was set up that the Phe79Tyr-Tyr80Phe-Asn192His triple variant has DDC-like activity, whereas the Asn192His and Phe79Tyr-Ty80Phe variants have both DHPAAS and DDC activities. To verify this hypothesis and obtain a comprehensive finding for the actions of DHPAAS, the enzymatic activities of wild-type DHPAAS, Asn192His variant, Phe79Tyr-Ty80Phe variant, and Phe79Tyr-Tyr80Phe-Asn192His DHPAAS variant of B. mori were evaluated (FIGS. 5 and 6).

From the ninhydrin staining after TLC, it was confirmed that the main product of the Phe79Tyr-Tyr80Phe-Asn192His DHPAAS variant was dopamine, supporting the hypothesis described above (FIG. 5B). When the product obtained by longer incubation was analyzed, THP was detected as the primary cation in the reaction product of L-DOPA and Phe79Tyr-Tyr80Phe-Asn192His DHPAAS (FIG. 5D).

The activity of DHPAAS was then evaluated with H₂O₂ production. H₂O₂ was quantified using a hydrogen peroxide fluorescence quantitation assay kit (Sigma) with a 96-hole plate. 0.6-0.8 μg of DHPAAS was dissolved in PBS (20 μL) and mixed with various concentrations of L-DOPA (10 μL) followed by the addition of 30 μL of a peroxidase enzyme mixture (Sigma). Fluorescence was detected using a SpectraMax Paradigm microplate reader (Molecular Devices). As a result, it was found that Asn192 was most important for maintaining the activity of DHPAAS, and Phe79 and Tyr80 also influenced the activity of DHPAAS (FIG. 6).

6. In Vitro THP Production by DHPAAS

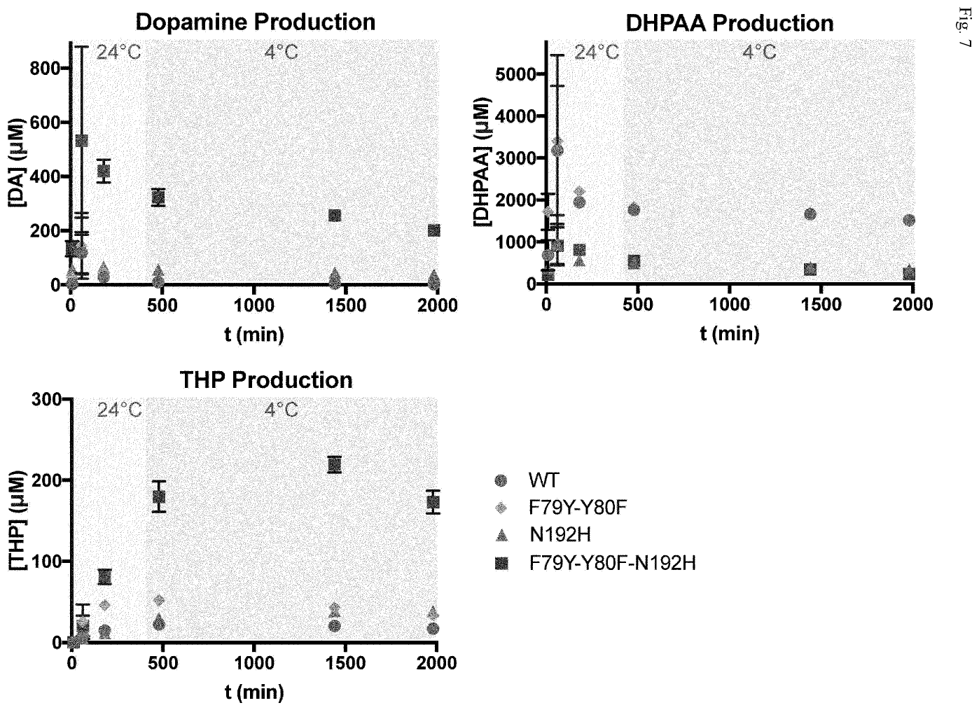

Since it was confirmed that THP could be produced directly by Phe79Tyr-Tyr80Phe-Asn192His DHPAAS, in vitro THP production was evaluated using the wild-type and designed three variants of B. mori DHPAAS (FIG. 7).

Specific test methods are as follows: DHPAAS (2 to 3 μg) dissolved in PBS was mixed with an aqueous L-DOPA solution such that the final volume was 40 μL. To this mixture, L-DOPA at a final concentration of 1.875 mM and sodium ascorbate at a final concentration of 2.5 mM were added. The reaction was started at room temperature (23-24° C.) and the temperature was set to 4° C. after 8 hours. 2 μL of the reaction solution was collected at various times and diluted with 98 μL of MeOH containing ascorbic acid and camphorsulfonic acid. The dilution reaction solution was immediately stored at −30° C. and stored until LC-MS analysis.

Productions of dopamine, DHPAA and THP were monitored using LC-MS manipulated in MRM mode. As shown by detection of oxidized THP ions m/z 284.10 and m/z 306.15, THP yields were extremely sensitive to oxidation. THP-quinone ([THP-3H]+=284.0917) corresponds to primary ion m/z 284.10. The identified cation m/z 306.15 may correspond to N-oxide of THP ([THP+OH]+=306.1336).

The THP yields in vitro was significantly improved when ascorbic acid was added to suppress oxidative degradation of the product by H₂O₂. When 2.5 mM sodium ascorbate was added, the conversion rate from L-DOPA to THP by the Phe79Tyr-Tyr80Phe-Asn192His DHPAAS variant increased to 23.9% (219 This exceeded the highest in vivo conversion rate from dopamine to THP of 15.9% (Nakagawa, A. et al. Sci. Rep. 4, 6695 (2014)). The fact that ascorbic acid did not inhibit DHPAA production by DHPAAS indicates that DHPAA is not a secondary product of dopamine by H₂O₂ oxidation, but a product of direct enzymatic reaction of L-DOPA.

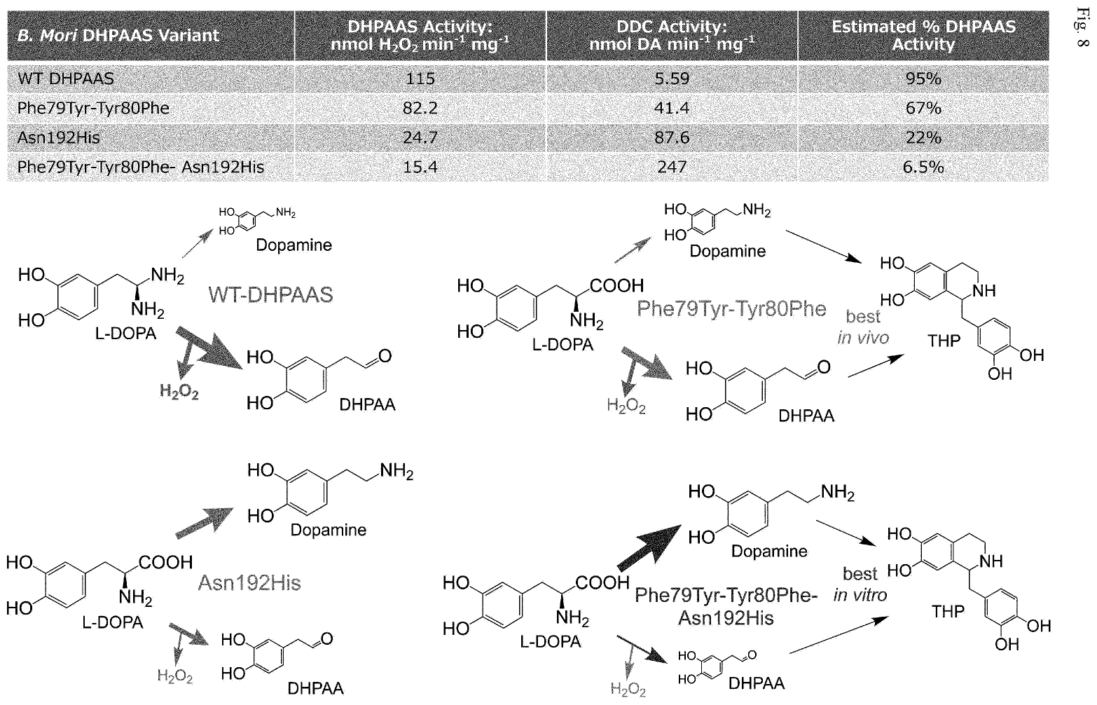

As predicted, the amount of DHPAA produced was the highest when the wild-type enzyme or Phe79Tyr-Tyr80Phe variant was used, and the lowest when the Asn192His variant or Phe79Tyr-Tyr80Phe-Asn192His variant was used (FIGS. 7 and 8). A reverse trend was observed in dopamine production as expected, and the amount produced was the highest when the Phe79Tyr-Tyr80Phe-Asn192His variant was used and the lowest when the wild-type DHPAAS was used, but dopamine production with the Asn192His variant was higher than with the Phe79Tyr-Tyr80Phe variant. The results of these in vitro tests support the hypothesis derived from the 3D structure with respect to the effects of Phe79, Tyr80, and Asn192 in the functional transformation of DHPAAS (FIG. 8).

7. In Vivo THP Production by DHPAAS

For cloning into an expression vector pTrcHis2B, the DHPAAS sequence was PCR amplified using a primer containing NcoI and XhoI restriction enzyme sites. The resulting untagged expression vector was introduced into BL21 (DE3) pLysS by transformation. For bioproduction, 3.5 mL of M9 medium containing 15.6 mM sodium ascorbate, 100 μg/mL ampicillin and 34 μg/mL chloramphenicol was used to grow E. coli at 37° C. with shaking at 200 rpm. When OD600 reached 0.2-0.4, IPTG was added at a final concentration of 0.97 mM to induce expression of DHPAAS, and the culture temperature was reduced to 25° C. After 1 hour and 13 minutes from induction, 3.4 mg of L-DOPA (0.97 mg/mL) was added to each culture, followed by the addition of PLP at a final concentration of 4.86 μM. After 12.9 hours from adding L-DOPA, the culture temperature was reduced to 16° C. Culture samples (300 to 500 μL) were collected at four time points and filtered through a Millipore Amicon Ultra 0.5 mL centrifuge filter with a molecular weight cutoff of 3,000 Da. After 22.7 hours from substrate addition, about 4 to 5 mg of ascorbic acid was added to each culture and the culture was transferred to 4° C. After 49.8 hours from substrate addition, the cultures were centrifuged at 4,500 g and the supernatant was collected for final measurement. The culture supernatant was diluted with MeOH to quantify L-DOPA, dopamine, DHPAA and THP.

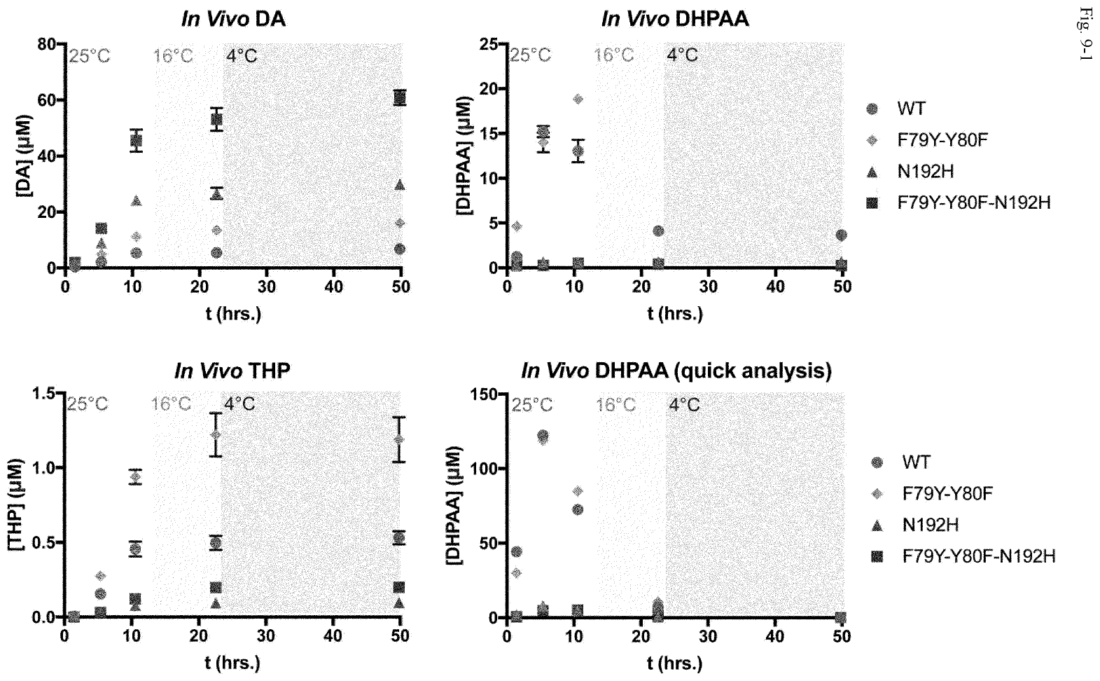

In an initial attempt with E. coli grown in LB medium, the amounts of THP produced were generally low, and here the amount produced with the Phe79Tyr-Tyr80Phe variant was slightly higher followed by the amount produced with the wild-type DHPAAS. However, when the medium was changed to M9 minimal medium, the amounts of THP produced increased considerably (FIG. 9).

Bioproduction of dopamine and DHPAA in vivo is perfectly consistent with the hypothesis based on the structure of DHPAAS, and is the consequence of substitutions of Phe79, Tyr80, and Asn192. In contrast to the results in vitro, the amount of THP produced with the Phe79Tyr-Tyr80Phe variant was 0.902 showing the strongest THP production in vivo. The wild-type DHPAAS showed the next highest amount produced, followed by the Phe79Tyr-Tyr80Phe-Asn192His DHPAAS, the Asn192His variant. As shown by chiral LC-MS analysis (FIG. 9-2), a diastereomeric mixture of (R, S)-THP was produced by DHPAAS in vivo.

8. In Vivo Production of Reticuline

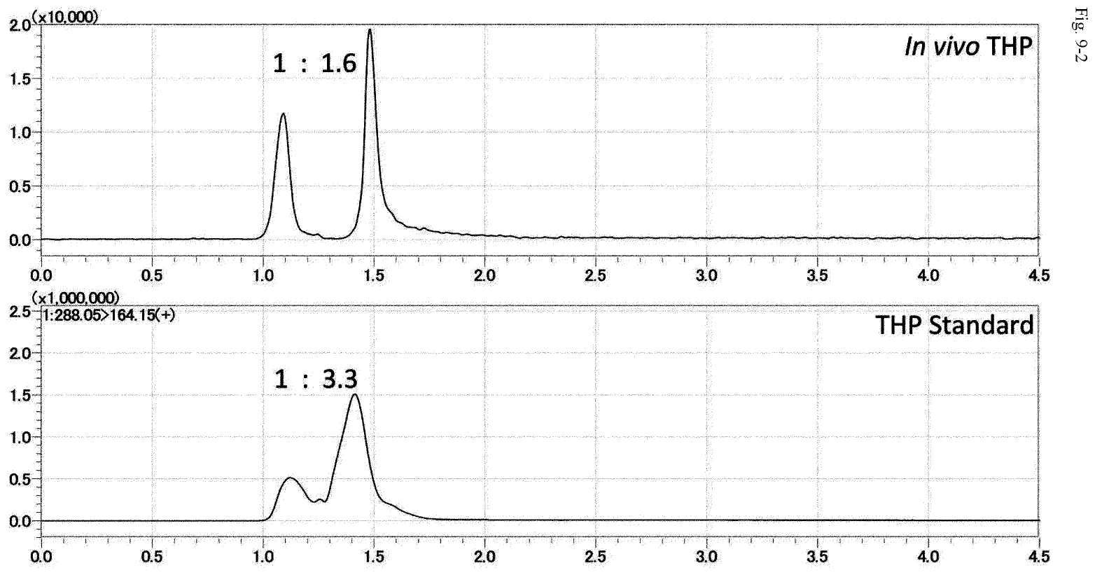

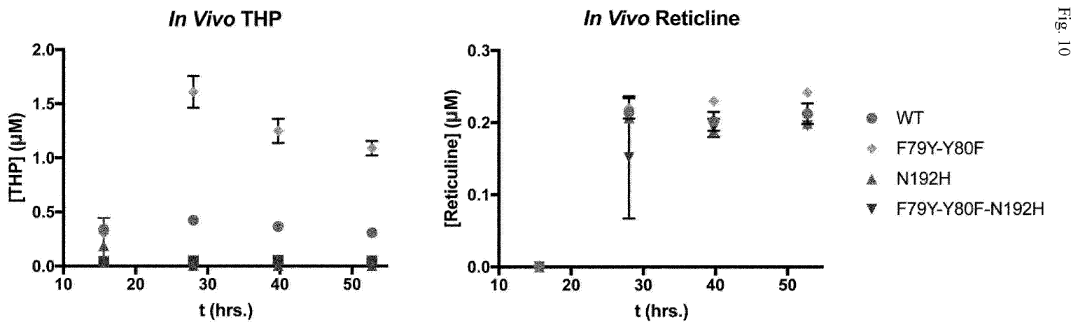

Simultaneously to the expression of DHPAAS, three enzymes for conversion from THP to reticuline were expressed in E. coli to confirm in vivo production of reticuline. Specifically, BL21 (DE3) pLysS was co-transformed with a pACYC184 vector (SEQ ID NO: 13) that expresses norcoclaurine 6-O-methyltransferase (6′OMT), 3′-hydroxy-N-methyl-(S)-coclaurine-4′-O-methyltransferase (4′OMT), and coclaurine-N-methyltransferase (CNMT) genes derived from C. japonica, and the DHPAAS expression vector derived from pTrcHis2B (SEQ ID NO: 14) obtained in Example 7, and the resulting reticuline-producing E. coli was selected with ampicillin and chloramphenicol. Reticuline production was tested in M9 minimal medium supplemented with 2% glucose. E. coli were grown until OD600 reached 0.2-0.3, and 0.5 mM IPTG 450 μM L-DOPA, and 4.54 mM sodium ascorbate were added thereto. After 17.2 hours from the substrate addition, 444 μM ascorbate was further added. E. coli were cultured at 25° C. with shaking at 200 rpm to produce reticuline. For quantifying dopamine, DHPAA, THP and reticuline, the culture was diluted with MeOH containing camphorsulfonic acid and ascorbic acid. Duplicate measurements were performed for Phe79Tyr-Tyr80Phe and Phe79Tyr-Tyr80Phe-Asn192His mediated reticuline productions, and four measurements were performed for wild-type and Asn192His mediated reticuline productions. The results are shown in FIG. 10.

9. In Vivo Production of THP, Reticuline, and Intermediates

BL21 (DE3) pLysS was co-transformed with an expression vector pTrcHis2B having the wild-type DHPAAS introduced therein, an expression vector pE-SUMO having the Phe79Tyr-Tyr80Phe-Asn192His variant DHPAA introduced therein, and a pACYC184 vector having norcoclaurine 6-O-methyltransferase (6′OMT), 3′-hydroxy-N-methyl-(S)-coclaurine-4′-O-methyltransferase (4′OMT), and coclaurine-N-methyltransferase (CNMT) genes derived from C. japonica introduced therein. In the first step of THP production, these three plasmid systems were cultured at 37° C. in glycerol-free TB supplemented with 1.5% glucose, 100 μg/mL ampicillin, and 50 μg/mL kanamycin. After OD600 reached 0.38, IPTG was added such that the final concentration was 0.5 mM. After 1.5 hours from induction, the temperature was reduced to 25° C. After 5.5 hours from induction, cells were collected by centrifugation at 4000×g, and stored at −80° C. overnight. Pellets from approximately 43 mL of the culture were resuspended in M9 containing low calcium, 0.2% Triton X-100, 1.5% glucose, 10 μM PLP, 10 mM sodium ascorbate, 1 mM L-DOPA such that the final volume was 6.5 mL. After mixing, the culture was maintained at 24-25° C. for 1.5 hours, and centrifuged at 5000×g to concentrate dopamine and DHPAA in the supernatant. After 25 hours from substrate addition, the supernatant was centrifuged again at 5000×g and the THP-containing supernatant was used for the next BIA production step.

In the second stage of BIA production, pET23a containing 4-OMT of C. japonica and 6-OMT and CNMT of P. somniferum was introduced into BL21 (DE3). This cell was initially cultured at 37° C. in glycerol-free TB supplemented with 1.5% glucose, 100 μg/mL ampicillin. After OD600 reached 0.78, IPTG was added such that the final concentration was 0.5 mM. After 1.5 hours from induction, the temperature was reduced to 25° C. After 5.5 hours from induction, cells were collected by centrifugation at 4000×g, and stored at −80° C. for 2 nights. Pellets from 46 mL of the culture were resuspended in the supernatant of the first step. Then, the amounts of BIA produced (3HC, 3HNMC, and reticuline) were measured while shaking at 25° C.

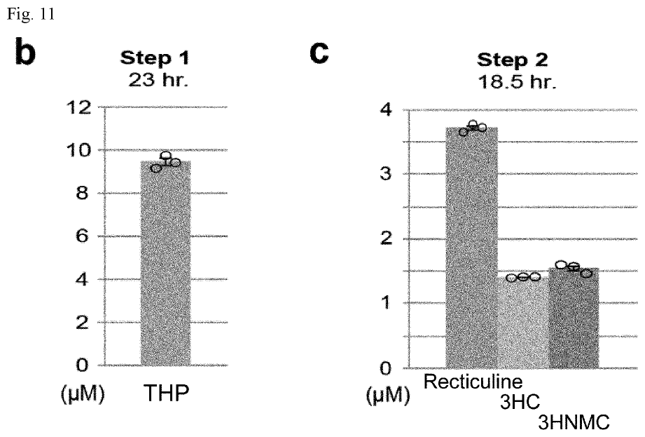

Diluted samples of the medium were analyzed using LC-MS and MRM. The results are shown in FIG. 11. Note that THP was quantified after 23 hours from L-DOPA addition, and 3HC, 3HNMC, and reticuline were quantified after 18.5 hours from addition of the second bioproducer (supernatant of the first step). Here, the error bars in the figure show standard errors of the mean (independent measurements of n=3 were performed).

As shown in FIG. 11, it was confirmed that THP, reticuline, and two intermediates were produced by the two-stage cell production system described above.

10. In Vivo THP Production (Introduction of Three Variants of DHPAAS and TfNCS)

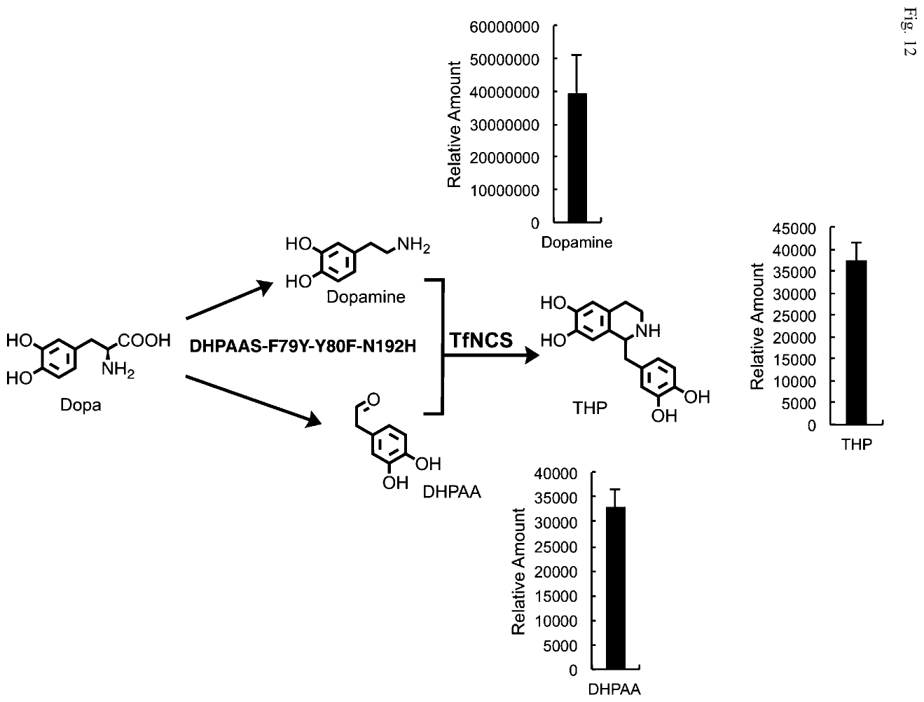

BL21 (DE3) was co-transformed with a Phe79Tyr-Tyr80Phe-Asn192His variant DHPAAS-expressing vector pTrcHis2B-tDHPAAS, and a NCS-expressing vector pCDFDuet-1-TfNCS. The cells were cultured at 37° C. in LB medium supplemented with ampicillin, spectinomycin, 1 mM ascorbic acid. After OD600 reached 0.4-0.6, IPTG was added such that the final concentration was 0.5 mM. After 3 hours, the cells were collected by centrifugation at 4000×g, and the pellets were resuspended in LB medium containing 135 μM. PLP, 5.1 mM sodium ascorbate, 1.97 mM L-DOPA, and 1.94 mM α-methyldopa. After mixing, the culture was maintained at 25° C. for 16.5 hours and centrifuged at 5000×g, and dopamine, DHPAA, and THP in the supernatant were quantified using LC-MS and MRM. The results are shown in FIG. 12.

As shown in FIG. 12, introduction of TfNCS resulted in collection of a large amount of THP, compare with the experiment with endogenous NCS alone.

11. In Vivo Production of Norcoclaurine from Tyrosine (Introduction of TyDC of P. somniferum and NCS)
(1) Tests with Introduction of TyDC1 and TfNCS

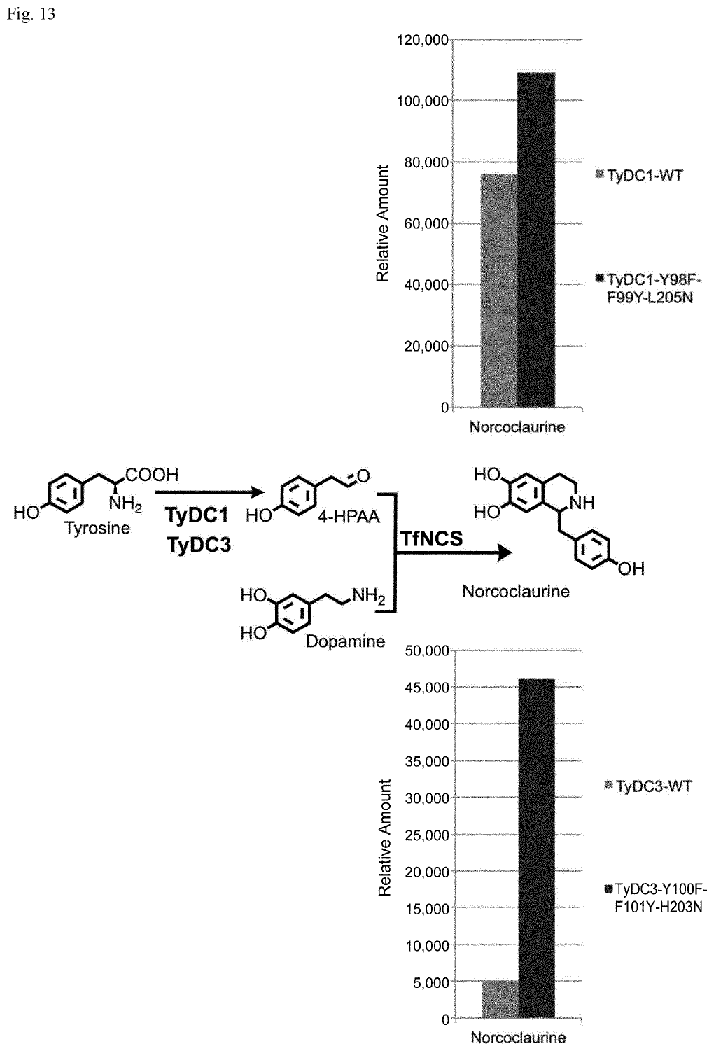

Various pCDFDuet-1-TfNCS-PsTyDC1 that are vectors into which a wild-type or a variant of TyDC1 (TyDC1-Y98F-F99Y-L205N) of P. somniferum and TfNCS (the codon-optimized nucleotide sequence is as shown in SEQ ID NO: 27 and the corresponding amino acid sequence is as shown in SEQ ID NO: 28) were introduced were generated. By using the primers of SEQ ID NOs: 17 and 18, a nucleotide into which mutations of Tyr98Phe, Phe99Tyr were introduced was synthesized. By using the primers of SEQ ID NO: 19 and SEQ ID NO: 20, a nucleotide into which a mutation of Leu205Asn was introduced was synthesized. Using the vector, BL21 (DE3) was transformed. The cells were cultured with shaking at 200 rpm at 37° C. in LB supplemented with spectinomycin. After OD600 exceeded 0.3, IPTG was added such that the final concentration was 0.5 mM, and the cells were cultured with shaking at 180 rpm at 28° C. After 1 hour, sodium ascorbate at a final concentration of 2 mM, dopamine (DA) at a final concentration of 0.5 mM, and tyrosine at a final concentration of 1 mM were added to the culture. After mixing, the cells were cultured with shaking for 51 hours, and norcoclaurine in the supernatant was quantified using LC-MS and MRM. The results are shown in FIG. 13.

(2) Tests with Introduction of TyDC3 and TfNCS

Various pCDFDuet-1-TfNCS-PsTyDC3 that are vectors into which a wild-type or a variant of TyDC3 (TyDC3-Y100E-F101Y-H₂O₃N) of P. somniferum and TfNCS (the codon-optimized nucleotide sequence is as shown in SEQ ID NO: 27 and the corresponding amino acid sequence is as shown in SEQ ID NO: 28) were introduced were generated. By using the primers of SEQ ID NOs: 23 and 24, a nucleotide into which mutations of Phe101Tyr, Tyr100Phe were introduced was synthesized. By using the primers of SEQ ID NO: 25 and SEQ ID NO: 26, a nucleotide into which a mutation of His203Asn was introduced was synthesized. Using the vector, BL21 (DE3) was transformed. The cells were cultured with shaking at 200 rpm at 37° C. in LB supplemented with spectinomycin. After OD600 exceeded 0.3, IPTG was added such that the final concentration was 0.5 mM, and the cells were cultured with shaking at 180 rpm at 28° C. After 1 hour, sodium ascorbate at a final concentration of 2 mM, dopamine (DA) at a final concentration of 0.5 mM, and tyrosine at a final concentration of 1 mM were added to the culture. After mixing, the cells were cultured with shaking for 51 hours, and norcoclaurine in the supernatant was quantified using LC-MS and MRM. The results are shown in FIG. 13.

(3) Tests with Introduction of TyDC1 and PSONCS3

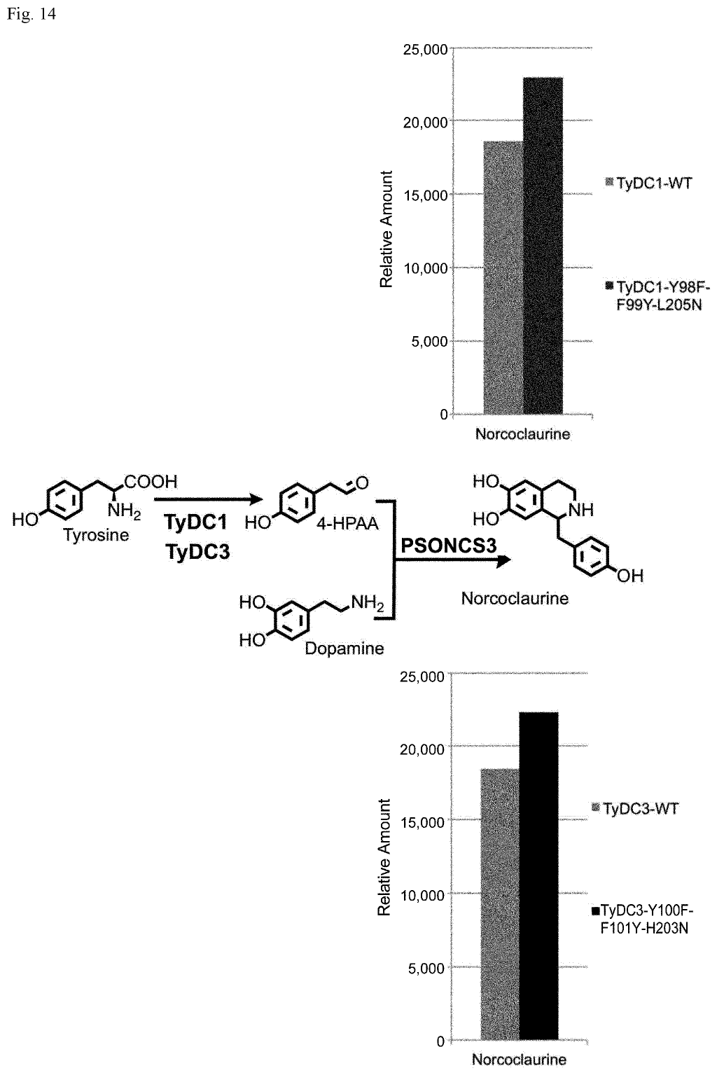

Various pCDFDuet-1-PSONCS3-PsTyDC1 that are vectors into which a wild-type or a variant of TyDC1 (TyDC1-Y98F-F99Y-L205N) of P. somniferum and PSONCS3 (the codon-optimized nucleotide sequence is as shown in SEQ ID NO: 29 and the corresponding amino acid sequence is as shown in SEQ ID NO: 30) were introduced were generated. By using the primers of SEQ ID NOs: 17 and 18, a nucleotide into which mutations of Phe99Tyr, Tyr98Phe were introduced was synthesized. By using the primers of SEQ ID NO: 19 and SEQ ID NO: 20, a nucleotide into which a mutation of Leu205Asn was introduced was synthesized. Using the vector, BL21 (DE3) was transformed. The cells were cultured with shaking at 200 rpm at 37° C. in LB supplemented with spectinomycin. After OD600 exceeded 0.3, IPTG was added such that the final concentration was 0.5 mM, and the cells were cultured with shaking at 180 rpm at 28° C. After 1 hour, sodium ascorbate at a final concentration of 2 mM, dopamine (DA) at a final concentration of 0.5 mM, and tyrosine at a final concentration of 1 mM were added to the culture. After mixing, the cells were cultured with shaking for 51 hours, and norcoclaurine in the supernatant was quantified using LC-MS and MRM. The results are shown in FIG. 14.

(4) Tests with Introduction of TyDC3 and PSONCS3

Various pCDFDuet-1-PSONCS3-PsTyDC3 that are vectors into which a wild-type or a variant of TyDC3 (TyDC3-Y100E-F101Y-H203N) of P. somniferum and PSONCS3 (the codon-optimized nucleotide sequence is as shown in SEQ ID NO: 29 and the corresponding amino acid sequence is as shown in SEQ ID NO: 30) were introduced were generated. By using the primers of SEQ ID NOs: 17 and 18, a nucleotide into which mutations of Phe101Tyr, Tyr100Phe were introduced was synthesized. By using the primers of SEQ ID NO: 19 and SEQ ID NO: 20, a nucleotide into which a mutation of His203Asn was introduced was synthesized. Using the vector, BL21 (DE3) was transformed. The cells were cultured with shaking at 200 rpm at 37° C. in LB supplemented with spectinomycin. After OD600 exceeded 0.3, IPTG was added such that the final concentration was 0.5 mM, and the cells were cultured with shaking at 180 rpm at 28° C. After 1 hour, sodium ascorbate at a final concentration of 2 mM, dopamine (DA) at a final concentration of 0.5 mM, and tyrosine at a final concentration of 1 mM were added to the culture. After mixing, the cells were cultured with shaking for 51 hours, and norcoclaurine in the supernatant was quantified using LC-MS and MRM. The results are shown in FIG. 14.

As shown in FIGS. 13 and 14, 4-HPAA and norcoclaurine were successfully produced from tyrosine by introducing TyDC1 or TyDC3 of P. somniferum and NCS (TfNCS of Thalictrum flavum or PSONCS3 of P. somniferum) into the cells. In addition, the amount of norcoclaurine produced could be significantly increased by introducing the mutation described above into TyDC1 or TyDC3. The 98th, 99th, 205th amino acids in each TyDC1 correspond to the 79th, 80th, 192th amino acids in DHPAAS having a common structure. In this study, mutations of 98th amino acid of TyDC1 from Tyr to Phe, 99th amino acid from Phe to Tyr, and 205th amino acid from His to Asn can be considered as a modification of these residues contributing to the carboxylase activity of TyDC1 to have AAS activity. The same holds true for TyDC3.

12. In Vivo Production of Reticuline from Tyrosine (Introduction of TyDC of P. somniferum and NCS)

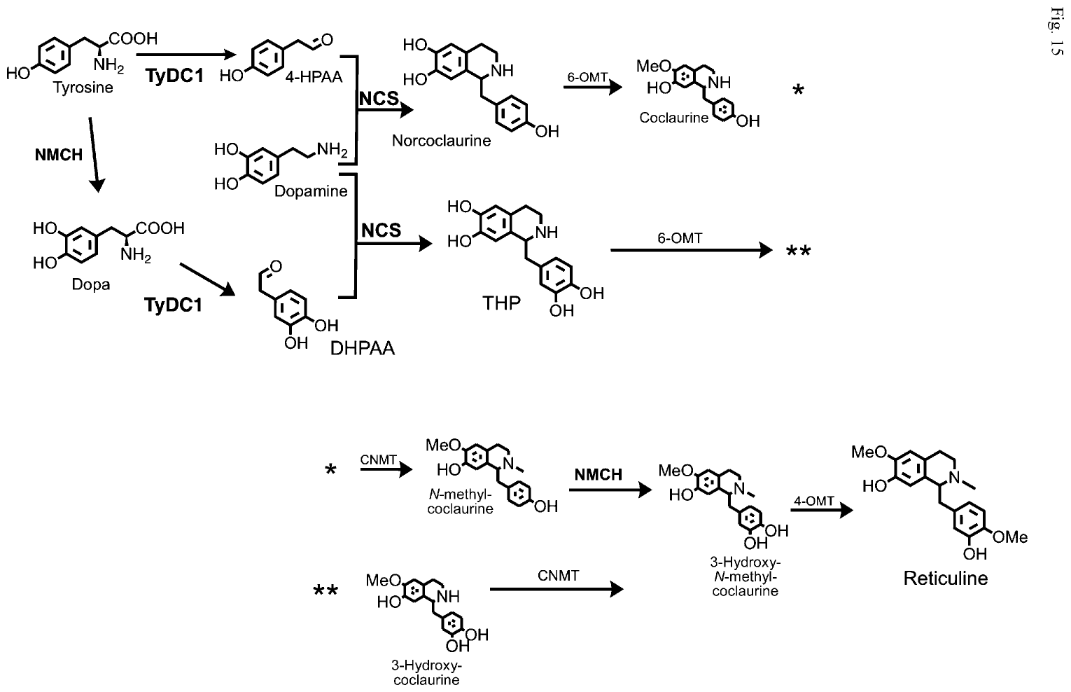

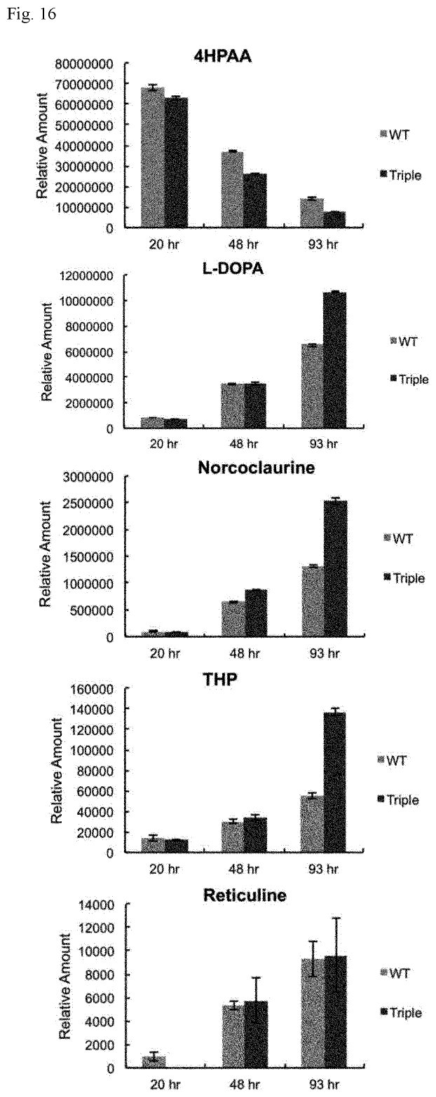

In the same manner as described in 11(1) above, various pCDFDuet-1-TfNCS-PsTyDC1 that are vectors into which a wild-type or a variant of TyDC1 (TyDC1-Y98F-F99Y-L205N) of P. somniferum and TfNCS were introduced were generated. Furthermore, a pACYC184 vector that expresses norcoclaurine 6-O-methyltransferase (6′OMT), 3′-hydroxy-N-methyl-(S)-coclaurine-4′-O-methyltransferase (4′OMT), coclaurine-N-methyltransferase (CNMT) genes derived from C. japonica, and N-methylcoclaurine 3-hydroxylase (NMCH) from P. somniferum, described in 9 above, was employed. Using these vectors, BL21 (DE3) was transformed. The cells were cultured with shaking at 180 rpm at 37° C. in M9 medium supplemented with spectinomycin, chloramphenicol, and 5 mM ascorbic acid. When OD600 reached 0.2-0.3, IPTG was added such that the final concentration was 0.8 mM, and the cells were cultured with shaking at 180 rpm at 25° C. for 30 minutes. Dopamine (DA) at a final concentration of 2.5 mM and tyrosine at a final concentration of 5 mM were added to the culture, and after mixing, the culture was cultured with shaking at 180 rpm for 93 hours. L-DOPA, 4HPAA, norcoclaurine, THP, and reticuline in the supernatant were quantified using LC-MS and MRM. The in vivo reaction schemes are shown in FIG. 15, and the amounts of L-DOPA, 4HPAA, norcoclaurine, THP, and reticuline produced in the supernatant are shown in FIG. 16.

As shown in FIG. 16, reticuline was finally successfully produced from tyrosine by introducing TyDC1 of P. somniferum, NCS, and further 6′OMT, 4′OMT, CNMT, NMCH into the cells.

13. In Vivo Production of THP and Reticuline from L-DOPA (Introduction of Modified DDC of P. putida)

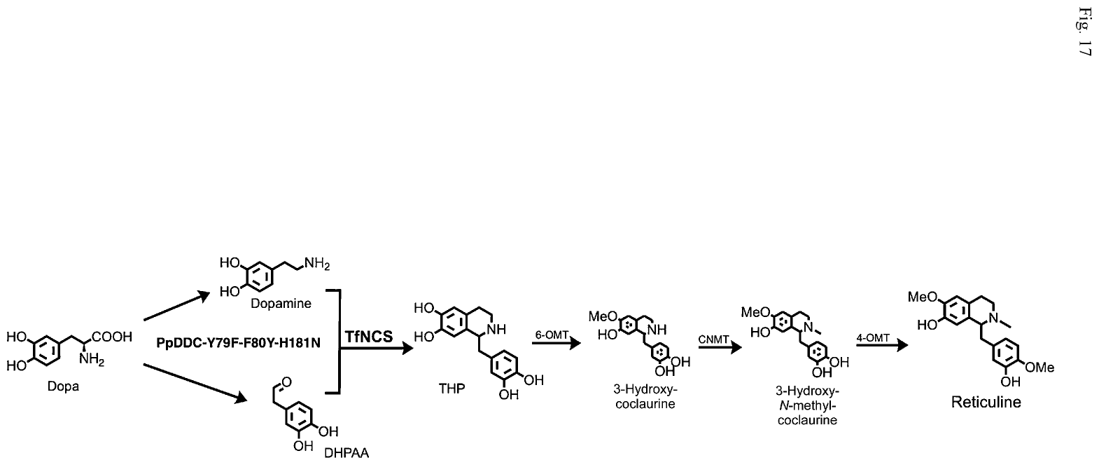

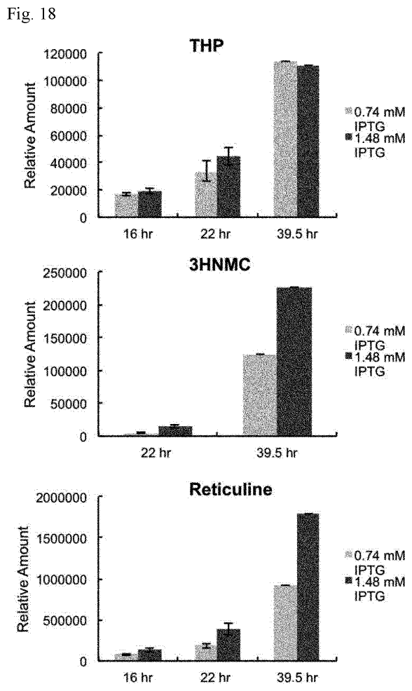

BL21 (DE3) was transformed with pACYC184 into which a variant of DDC (DDC-Y79F-F80Y-H₁₈₁N) of P. putida, norcoclaurine 6-O-methyltransferase (6′OMT), 3′-hydroxy-N-methyl-(S)-coclaurine-4′-O-methyltransferase (4′OMT), and coclaurine-N-methyltransferase (CNMT) derived from C. japonica were introduced. The cells were cultured with shaking at 180 rpm at 28° C. in LB medium supplemented with spectinomycin and chloramphenicol. When OD600 exceeded 0.3, IPTG was added such that the final concentration was 0.74 mM to 1.48 mM. The cells were cultured at 20° C., 180 rpm for 30 minutes. L-DOPA at a final concentration of about 1.9 mM and sodium ascorbate at a final concentration of about 4.7 mM were added to the culture, and after mixing, the cells were cultured for 40 hours. THP and reticuline in the supernatant were quantified using LC-MS and MRM. The in vivo reaction schemes are shown in FIG. 17, and the amounts of THP, 3HNMC, and reticuline produced in the supernatant are shown in FIG. 18.

As shown in FIGS. 17 and 18, THP, 3HNMC and reticuline could be produced from L-DOPA by a variant of DDC (DDC-Y79F-F80Y-H181N) of P. putida. This is a result indicating that the variant of DDC (DDC-Y79F-F80Y-H181N) was able to induce both dopamine and DHPAA from L-DOPA. Thus, in the tests described above, DHPAAS activity was successfully generated in DDC by introducing a reverse mutation (Tyr79Phe-Phe80Tyr-His181Asn) to the mutation introduced in DHPAA (Phe79Tyr-Tyr80Phe-Asn192His) into the DDC of P. putida.

### INDUSTRIAL APPLICABILITY

According to the present invention, by using a recombinant host cell expressing a wild-type or a variant of an aromatic aldehyde synthase (AAS) or an aromatic amino acid decarboxylase (AAAD), which are difunctional enzymes, a benzylisoquinoline alkaloid (BIA) can be efficiently and easily produced.

1. A recombinant host cell for producing a benzylisoquinoline alkaloid (BIA), expressing a wild-type or a variant of an aromatic aldehyde synthase (AAS) and an aromatic amino acid decarboxylase (AAAD) of a heterologous species.

2. The recombinant host cell according to claim 1, wherein the benzylisoquinoline alkaloid (BIA) is tetrahydropapaveroline (THP), norcoclaurine, 3-hydroxycoclaurine, 3-hydroxy-N-methylcoclaurine and/or reticuline.

3. The recombinant host cell according to claim 1, wherein the heterologous species is an insect, a plant, or a microorganism.

4. The recombinant host cell according to claim 3, wherein the heterologous species is an insect selected from the group consisting of Bombyx mori, Camponotus floridanus, Apis mellifera, Aedes aegypti and Drosophila melanogaster, Papaver somniferum, or Pseudomonas putida.

5. The recombinant host cell according to claim 1, wherein the host cell is E. coli.

6. The recombinant host cell according to claim 1, wherein the aromatic aldehyde synthase (AAS) is 3,4-dihydroxyphenylacetaldehyde synthase (DHPAAS) or 4-hydroxyphenylacetaldehyde synthase (4-HPAAS).

7. The recombinant host cell according to claim 6, wherein the aromatic aldehyde synthase (AAS) is derived from an insect and a mutation in the variant of the aromatic aldehyde synthase (AAS) is at least one selected from the group consisting of Asn192His, Phe79Tyr and Tyr80Phe.

8. The recombinant host cell according to claim 6, wherein the aromatic amino acid decarboxylase (AAAD) is tyrosine decarboxylase (TyDC) derived from a plant, and a mutation in the variant of the tyrosine decarboxylase (TyDC) is at least one selected from the group consisting of Leu205Asn, Phe99Tyr and Tyr98Phe, or at least one selected from the group consisting of His203Asn, Phe101Tyr and Tyr100Phe.

9. The recombinant host cell according to claim 6, wherein the aromatic amino acid decarboxylase (AAAD) is dopa decarboxylase (DDC) derived from a microorganism, and a mutation in the variant of the dopa decarboxylase (DDC) is at least one selected from the group consisting of Tyr79Phe, Phe80Tyr and His181Asn.

10. The recombinant host cell according to claim 1, further expressing norcoclaurine synthase (NCS).

11. The recombinant host cell according to claim 1, further expressing at least one enzyme selected from the group consisting of norcoclaurine 6-O-methyltransferase (6′OMT), 3′-hydroxy-N-methyl-(S)-coclaurine-4′-O-methyltransferase (4′OMT), coclaurine-N-methyltransferase (CNMT) and N-methylcoclaurine 3-hydroxylase.

12. A method for producing a benzylisoquinoline alkaloid (BIA), comprising a step of culturing the recombinant host cell according to claim 1 in a L-DOPA or tyrosine-containing culture medium.

13. A method for producing a benzylisoquinoline alkaloid (BIA), comprising a step of causing a wild-type or a variant of an aromatic aldehyde synthase (AAS), an aromatic amino acid decarboxylase (AAAD) to act on L-DOPA or tyrosine in a cell-free system.

14. A method for producing a benzylisoquinoline alkaloid (BIA), comprising a step of causing a wild-type or a variant of an aromatic aldehyde synthase (AAS), an aromatic amino acid decarboxylase (AAAD) to act on L-DOPA or tyrosine in a cell-free system, wherein the wild-type or the variant of the aromatic aldehyde synthase (AAS), the aromatic amino acid decarboxylase (AAAD) is an enzyme obtained from the recombinant host cell according to claim 1.
